(19) United States
(12) Patent Application Publication
(10) Pub. No.: US 2024/0041927 A1
(43) Pub. Date: Feb. 8, 2024

(54) CHIMERIC AUTOANTIBODY RECEPTOR (CAAR) COMPRISING A NICOTINIC ACETYLCHOLINE RECEPTOR AUTOANTIGEN

(71) Applicant: DEUTSCHES ZENTRUM FÜR NEURODEGENERATIVE ERKRANKUNGEN E. V. (DZNE), Bonn (DE)

(72) Inventors: Niels VON WARDENBURG, Berlin (DE); Harald PRÜSS, Berlin (DE); Marie Alice HOMEYER, Hennigsdorf (DE); Momsen S. REINCKE, Berlin (DE)

(73) Assignee: DEUTSCHES ZENTRUM FÜR NEURODEGENERATIVE ERKRANKUNGEN E. V. (DZNE), Bonn (DE)

(21) Appl. No.: 18/268,803

(22) PCT Filed: Dec. 22, 2021
(86) PCT No.: PCT/EP2021/087201

§ 371 Date: Jun. 21, 2023

(30) Foreign Application Priority Data
Dec. 22, 2020 (EP) 20216515.5

(51) Int. Cl. A61K 35/17; C07K 14/705

(52) U.S. Cl. CPC A61K 35/17; C07K 14/70578; C07K 14/70517; C07K 2319/03; A61K 38/00

(57) ABSTRACT

The invention relates to a chimeric autoantibody receptor (CAAR) and nucleic acid molecules encoding said CAAR, wherein the CAAR comprises an extracellular domain comprising an autoantigen of a nicotinic acetylcholine receptor (nAChR) or fragment thereof. The invention relates further to a vector comprising a nucleic acid molecule encoding the CAAR, to a CAAR polypeptide, to a genetically modified cell expressing the CAAR or comprising a nucleic acid molecule or a vector encoding the CAAR. The invention relates further to genetically modified cells expressing the CAAR for use in the treatment of a neuromuscular disorder associated with autoantibodies that bind a nicotinic acetylcholine receptor (nAChR), preferably for the treatment of myasthenia gravis (MG).

The invention relates to the field of targeted cellular therapy employing a chimeric autoantibody receptor (CAAR) and the treatment of autoimmune neuromuscular disorders, such as myasthenia gravis (MG).

The invention relates to a chimeric autoantibody receptor (CAAR) and nucleic acid molecules encoding said CAAR, wherein the CAAR comprises an extracellular domain comprising an autoantigen of a nicotinic acetylcholine receptor (nAChR) or fragment thereof. The invention relates further to a vector comprising a nucleic acid molecule encoding the CAAR, to a CAAR polypeptide, to a genetically modified cell expressing the CAAR or comprising a nucleic acid molecule or a vector encoding the CAAR. The invention relates further to genetically modified cells expressing the CAAR for use in the treatment of a neuromuscular disorder associated with autoantibodies that bind a nicotinic acetylcholine receptor (nAChR), preferably for the treatment of myasthenia gravis (MG).

### BACKGROUND OF THE INVENTION

Myasthenia gravis (MG) is an autoimmune disease caused by autoantibodies directed against the acetylcholine receptor (AChR) or other proteins in the postsynaptic neuromuscular endplate (Gilhus et al. 2019). The primary symptom of myasthenia gravis is localized or generalized muscle weakness, induced by autoantibodies. Autoantibodies against AChR can be detected in about 90% of cases of myasthenia gravis. With an annual incidence of 8 to 10 cases per 1 million individuals and a prevalence of 150 to 250 cases per 1 million, myasthenia gravis is one of the most significant neuromuscular autoimmune diseases (Gilhus et al. 2016). The removal of antibodies from patients' blood leads to a significant clinical improvement, allowing many patients to lead independent lives again after a prolonged course of the disease. However, more severe forms of the disease regularly lead to chronically elevated antibody levels and myasthenic crises, which require intensive care monitoring and mechanical ventilation.

A fundamental problem in MG treatment is that the removal of anti-AChR antibodies (e.g. by means of blood apheresis) and immunosuppression (e.g. with prednisolone, azathioprine or rituximab to remove the antibody-producing B-cells), which both lead to an improvement in patients' condition, are associated with considerable side effects (Gilhus et al. 2016). This is particularly true for severe forms of myasthenic crisis, where, for example, plasmapheresis is necessary. Possible complications of plasmapheresis are injuries through the central venous catheter, circulatory regulation disorders due to fluid shifts, coagulation disorders with thromboses and infections, including potentially sepsis.

Potential side effects of drug immunosuppression are a susceptibility to severe infections, as well as the sometimes considerable side effects of long-term therapy with steroids, such as an increased cardiovascular risk profile or weight gain triggered by the medication. Furthermore, vaccinations and the body's own defense mechanisms are dependent on antibodies, which are removed or depleted by unspecific immunotherapy.

Eculizumab has been approved for severe forms of myasthenia gravis since 2017. Although this therapy is advertised with a presumably low side effect profile, it has other disadvantages due to the necessity of regular, two-week infusions as well as annual therapy costs of currently approx. 500,000 Euro. This therapy also does not act upon the underlying pathogenesis of the disease, namely the disease-inducing autoantibodies, but only at the complement-mediated endpoint of the disease mechanism.

This problem can only be solved by selectively removing disease-specific AChR autoantibodies with as few side effects as possible. So far, no therapeutic procedure is available for MG that works according to this principle. The present invention therefore seeks to address this significant problem by employing chimeric autoantibody receptor (CAAR) expressing-cells binding AChR autoantibodies.

Chimeric antigen receptor (CAR) T cells are human T cells that have been genetically modified to express a CAR, such that their activation does not occur via the normally occurring binding to MHC-presented peptides, but via a recombinant antibody or fragment thereof of the CAR located on the surface of the T cell. Until now, CAR-T approaches are primarily used in cancer therapy, where they recognize tumor-specific epitopes via a recombinant antibody portion of the CAR and selectively kill tumor cells through T cell activation.

The present invention however employs a chimeric autoantibody receptor (CAAR) expressed preferably from engineered T cells (CAAR-T cells), wherein the CAAR comprises—as a targeting domain in place of an antibody fragment—an autoantigen that is bound by autoantibodies, which are evident in autoimmune neuromuscular disorders and presented by disease-causing B cells.

Chimeric autoantibody receptors (CAAR) are as such known in the art. Ellebrecht et al. (2016, Science) and WO 2015/168613 describe a CAAR-T approach directed against autoantibodies that bind the skin cell adhesion protein desmoglein 3 (Dsg3). Richman et al (NIH grant application 9600548) have also proposed chimeric autoantibody receptor (CAAR)-expressing T cells (CAART) to attack autoantibody-producing B cells in a rat model of muscle-specific kinase (MuSK)-MG experimental autoimmune MuSK myasthenia (EAMM). WO2019236593A1 discloses a chimeric autoantibody receptor (CAAR) specific for anti-muscle-specific kinase (MuSK) B cell receptor (BCR). WO2018127585 teaches chimeric autoantibody receptors (CAARs) specific for autoantibody-producing B-cells, for which various autoantigens are described. WO2019213434A1 discloses a chimeric autoantibody receptor (CAAR) comprising a phospholipase A2 receptor (PLA2R) autoantigen.

Thus, the present invention addresses the problems of unwanted unspecific immune-depletion and immunosuppression in treating autoimmune neuromuscular disorders. Although a number of potential alternatives for treating other autoimmune diseases are established or in development, a significant need remains for providing effective means for addressing this problem.

### SUMMARY OF THE INVENTION

In light of the prior art the technical problem underlying the invention was the provision of alternative or improved means for treating and/or preventing autoimmune neuromuscular disorders, such as myasthenia gravis (MG). A further objective of the invention was to provide therapeutic options that avoid or minimize unspecific immunosuppression.

This problem is solved by the features of the independent claims. Preferred embodiments of the present invention are provided by the dependent claims.

Therefore, the invention relates to a nucleic acid molecule encoding a chimeric autoantibody receptor (CAAR), wherein the nucleic acid molecule encodes:
an extracellular domain comprising an autoantigen of a nicotinic acetylcholine receptor (nAChR) or fragment thereof,
a transmembrane domain, and
an intracellular signaling domain.

The invention also relates to a chimeric autoantibody receptor (CAAR) polypeptide, for example encoded by the nucleic acid molecule of the invention, the CAAR polypeptide comprising:
an extracellular domain comprising an autoantigen of a nicotinic acetylcholine receptor (nAChR) or fragment thereof,
a transmembrane domain, and
an intracellular signaling domain.

In one embodiment, the invention relates to a nucleic acid molecule encoding a chimeric autoantibody receptor (CAAR), wherein the nucleic acid molecule encodes:
an extracellular domain comprising an autoantigen of a nicotinic acetylcholine receptor (nAChR), wherein the autoantigen comprises or consists of a beta-1 subunit of the nicotinic acetylcholine receptor (nAChR), or an autoantigenic fragment or variant thereof,
a transmembrane domain, and
an intracellular signaling domain.

In one embodiment, the invention relates to a chimeric autoantibody receptor (CAAR) polypeptide, for example encoded by the nucleic acid molecule of the invention, the CAAR polypeptide comprising:
an extracellular domain comprising an autoantigen of a nicotinic acetylcholine receptor (nAChR) or fragment thereof, wherein the autoantigen comprises or consists of a beta-1 subunit of the nicotinic acetylcholine receptor (nAChR), or an autoantigenic fragment or variant thereof,
a transmembrane domain, and
an intracellular signaling domain.

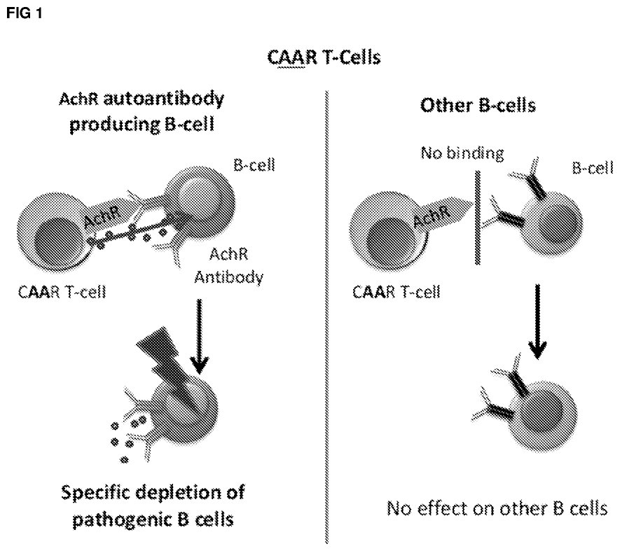

The difference of the present invention to the traditional CAR approach is that a receptor fragment of the nAChR is used instead of an antibody fragment, expressed as part of the CAR on the T cell surface (refer FIG. 1). When an nAChR autoantibody-producing B cell binds with its B cell receptor (via the antibody it produces) to the CAAR-T construct, the binding leads to an activation of the T cell, the formation of an ‘immunological synapse’ with the release of toxic mediators, which lead to the lysis of the disease-specific B cell (FIG. 1, left side). Other B cells (e.g. those producing antibodies after vaccination) are spared from depletion (FIG. 1, right side). In this way, the current invention solves the problems of unwanted unspecific immunodepletion or immune suppression.

The CAAR of the present invention represents an advantageous autoantibody-specific cellular immunotherapy approach towards treating autoimmune neuromuscular disorders employing an autoantigen of a nicotinic acetylcholine receptor (nAChR). It was surprising that the autoantigen-comprising constructs described herein would exhibit such beneficial autoantibody-specific B-cell depletion. To the knowledge of the inventors, the CAAR of the present invention represents the first autoantibody-specific cellular immunotherapy approach towards treating autoimmune neuromuscular disorders employing a beta-1 subunit autoantigen of a nicotinic acetylcholine receptor (nAChR).

The present invention leads to a number of fundamental improvements and advantages over treatments described in the prior art, for example the CAAR as described herein, and associated aspects of the inventions including corresponding CAAR modified immune cells, enable a selective and potentially curative approach towards treating the autoimmune neuromuscular disorders described herein. The autoantibody specificity achieved by incorporating—as a targeting domain for the CAAR-modified immune cells—an autoantigen of a nicotinic acetylcholine receptor (nAChR), which is bound by autoantibodies in autoimmune neuromuscular disorders, leads to selective removal of the disease agent with little or no widespread immunosuppression.

Furthermore, the elimination of the autoantibody producing B cells represents a potentially curative effect, such that the underlying cause of the disease agent is removed, thereby addressing the disease at the level of causality and leading to enhanced chances of long term or permanent mitigation of the disease. This combination of benefits represents an unexpectedly effective approach with a low risk profile regarding potential side effects due to widespread immunosuppression or disease recurrence.

Disadvantages of the prior art MG therapy relate to, for example, non-specific immunosuppression, only short-term effects of therapy, severe side effects, the necessity for multiple treatment cycles (e.g. 21 days of blood apheresis, or monthly chemotherapy), and high costs of the most effective drugs (for comparison: Eculizumab annual therapy costs are approx. 500,000 EUR).

Advantages of the present invention are, without limitation, a highly selective removal of nAChR-antibodies, long-term depletion of antibody-producing cells, no toxic side effects, potential immunological reactions are easily treatable, immediate (within hours) depletion of B-cells, potentially single administration of the cells (e.g. i.v.), and likely lower costs of CAAR-T-cells due to single treatment (single treatment with approved CAR-T-cell such as Kymriah are approx. 350,000 EUR).

The specific autoantigens employed in the constructs described herein therefore represent a novel and inventive group of autoantigens, derived from a nicotinic acetylcholine receptor (nAChR), which is targeted by autoantibodies in autoimmune neuromuscular disorders such as myasthenia gravis. A skilled person is capable of electing a suitable autoantigen from a nicotinic acetylcholine receptor (nAChR), for example by electing nAChR sequences of a preferably extracellular domain of the receptor known to be a target of autoantibodies in diseases such as myasthenia gravis. For example, the presence of serum or cerebrospinal fluid (CSF) autoantibodies to any given region of the nAChR indicates the suitableness of the autoantigen in the present invention.

In one embodiment, the nicotinic acetylcholine receptor (nAChR) autoantigen of the CAAR is bound by autoantibodies associated with a neuromuscular disorder.

In one embodiment, the autoantigen of the CAAR is bound by autoantibodies in subjects with myasthenia gravis (MG), or arthrogryposis multiplex congenita (AMC) caused by diaplacental transfer of autoantibodies.

In myasthenia gravis (MG) autoantibodies target key molecules at the neuromuscular junction, such as the nicotinic acetylcholine receptor (AChR), muscle-specific kinase (MuSK), low-density lipoprotein receptor-related protein 4 (Lrp4), Agrin and ColQ. These autoantibodies lead to a range of different pathogenic mechanisms to altered tissue architecture and reduced densities or functionality of AChRs, reduced neuromuscular transmission, and therefore a severe fatigable skeletal muscle weakness.

While most AChR antibodies target the AChR alpha subunit, there are individuals with antibodies against the fetal gamma subunit. The gamma subunit is only expressed during the first 30 weeks of life, after which it is exchanged by the adult epsilon subunit with the exception of the extraocular muscle, where AChR gamma subunit expression is maintained (Koneczny et al; Cells. 2019 July; 8(7): 671). Therefore, adults with these antibodies do not typically develop MG. When a healthy, pregnant woman produces anti-gamma subunit antibodies, they can be transferred through the placenta to the embryo. Here, the antibodies cause a fetal AChR inactivation syndrome, which leads to the reduced movement of the fetus. This has dire developmental consequences, the new-born children present with arthrogryposis multiplex congenita, a developmental disorder hallmarked by multiple joint contractures and profound respiratory impairment that may lead to severe disabilities, such as caused arthrogryposis multiplex congenita (AMC), or fetal death.

In one embodiment, the autoantigen of the CAAR comprises or consists of an extracellular part of the nicotinic acetylcholine receptor (nAChR) or fragment thereof bound by autoantibodies (autoantigenic fragment).

AChRs are members of a superfamily of neurotransmitter-gated ion channels, each comprised of five homologous subunits arranged around a central ion channel. Approximately 85% of MG patients have autoantibodies against the AChR. These antibodies mainly belong to the IgG1 and IgG3 subclass and many recognize the main immunogenic region (MIR) of the extracellular portion of the AChR alpha subunit. The extracellular N-terminal region of AChR alpha subunit represents the most common immunogenic region of the protein, although autoantibodies against other subunits have been identified. For example, autoantibodies against the beta-1 subunit are considered potentially relevant in autoimmune neuromuscular disorders such as MG. Despite the presence of autoantibodies directed to AChR in MG being known in the art, the present CAAR approach represents a surprisingly efficacious and beneficial approach in targeting the disease. It could not have been expected that the nAChR antigens employed herein lead to effective depletion of autoantibody-producing pathogenic B cells and potential amelioration of the disease. MG is defined by autoantibody responses to various antigenic targets and it was unexpected that the selection of antigens employed in the CAAR herein would be effective.

In one embodiment, the autoantigen of the CAAR comprises or consists of a beta-1, alpha-1, gamma, delta, or epsilon subunit of a nicotinic acetylcholine receptor (nAChR), or an autoantigenic fragment and/or combinations thereof, optionally comprising a linker.

In one embodiment, the autoantigen of the CAAR comprises or consists of a beta-1 subunit of a nicotinic acetylcholine receptor (nAChR), or an autoantigenic fragment thereof, optionally comprising a linker.

In one embodiment, the autoantigen of the CAAR comprises or consists of an alpha-1 subunit of a nicotinic acetylcholine receptor (nAChR), or an autoantigenic fragment thereof, optionally comprising a linker.

In one embodiment, the autoantigen of the CAAR comprises or consists of a subunit of a nicotinic acetylcholine receptor (nAChR) that is not an alpha-1 subunit, or an autoantigenic fragment thereof.

The AChR autoantibodies induce pathogenicity by three main mechanisms, namely (1) cross-linking and increased turnover of AChR, leading to reduced AChR levels at the NMJ, (2) activation of the classical complement cascade, formation of the membrane attack complex (MAC) and complement-mediated damage of the postsynaptic membrane, and (3) direct blocking of function by preventing the binding of acetylcholine to the receptor (Koneczny et al; Cells. 2019 July; 8(7): 671). By employing the autoantigenic portions of the nAChR, selected from the alpha-1, beta-1, gamma, delta, or epsilon subunits, autoantibody-producing B cells can be depleted and one or more of these pathogenic mechanisms can be countered.

In one embodiment, the autoantigen of the CAAR comprises or consists of a nicotinic acetylcholine receptor (nAChR) beta-1 subunit isoform 1 (SEQ ID NO: 3), beta-1 subunit isoform 2 (SEQ ID NO: 4), alpha-1 subunit isoform 1 (SEQ ID NO: 1), alpha-1 subunit isoform 2 (SEQ ID NO: 2), gamma subunit isoform 1 (SEQ ID NO: 5), gamma subunit isoform 2 (SEQ ID NO: 6), delta subunit isoform 1 (SEQ ID NO: 7), delta subunit isoform 2 (SEQ ID NO: 8), epsilon subunit (SEQ ID NO: 9), or an autoantigenic fragment and/or combination and/or variant with at least 80% sequence identity thereto, optionally comprising a linker.

In one embodiment, the autoantigen of the CAAR comprises or consists of a nicotinic acetylcholine receptor (nAChR) beta-1 subunit isoform 1 (SEQ ID NO: 3) or an autoantigenic fragment and/or variant with at least 80% sequence identity thereto, optionally comprising a linker.

In one embodiment, the autoantigen of the CAAR comprises or consists of an extracellular domain of a nicotinic acetylcholine receptor (nAChR) beta-1 subunit isoform 1 (SEQ ID NO: 21) or an autoantigenic fragment and/or variant with at least 80% sequence identity thereto, optionally comprising a linker.

In one embodiment, the autoantigen of the CAAR comprises or consists of a nicotinic acetylcholine receptor (nAChR) alpha-1 subunit isoform 1 (SEQ ID NO: 1) or an autoantigenic fragment and/or variant with at least 80% sequence identity thereto, optionally comprising a linker.

These antigen sequences relate to preferred sequences corresponding to the designated subunits that are targeted by pathologic autoantibodies in MG. Sequence variation of these preferred sequences is encompassed by the invention. Pathogenic autoantibodies are capable of binding sequence variants with for example at least 80% identity to the specific recited sequences, as some sequence variation may not change structural epitopes of the indicated autoantigens. Combinations of the mentioned sequences are also envisaged, as such constructs would potentially enable a greater number antibody-producing B cells to be targeted by the cells expressing the inventive CAAR. Linkers between potential combined antigen sequences are envisaged and examples are provided below. A skilled person is capable of designing sequence variants, combinations of antigenic sequences and selecting suitable linker sequences in order to arrive at a functional CAAR construct of the invention.

In one embodiment, the autoantigen of the CAAR comprises or consists of an extracellular autoantigenic part of an alpha-1 subunit isoform 1 (SEQ ID NO: 10), a combination of extracellular autoantigenic parts of alpha-1 isoform 1 and beta-1 isoform 1 subunits (SEQ ID NO: 11) or an extracellular autoantigenic part of a gamma subunit isoform 1 (SEQ ID NO: 12) of a nicotinic acetylcholine receptor (nAChR), or variant with at least 80% sequence identity thereto.

These antigens are preferred, also due to their practical exemplification in the examples below. The specific autoantigens disclosed herein are associated with advantages with respect to surprisingly good transduction rates and/or surface expression in transduced cells expressing the inventive CAAR, in addition to excellent activation of the modified T cells, when expressing the inventive CAAR, after stimulation with pathogenic autoantibodies.

Methodologies for determining autoantigens and relevant epitopes from the nAChR are known to a skilled person. Cell-based assays, or immunohistochemistry of unfixed sections, or similar methodologies used under various experimental conditions may be employed. Different portions of the autoantigen may be employed, and different immunoglobulins may be detected (such as, without limitation, IgG, IgA, and/or IgM).

The CAAR constructs of the present invention exhibit further unexpected and advantageous properties. For example, T-cells transduced with the inventive CAAR show only a minor reduction of killing efficiency when soluble nAChR-reactive antibodies are present in cell culture medium. This demonstrates that the cytotoxic T cells, once modified with the CAAR, are not rendered ineffective by soluble antibodies, and maintain effectiveness against B cells presenting pathogenic autoantibodies. In one embodiment of the invention, the CAAR-expressing cell, such as a T cell, maintains cytotoxic activity against target cells presenting unwanted autoantibodies in the presence of soluble reactive antibodies.

In some embodiments, the CAAR constructs encode (and the CAAR polypeptides comprise accordingly) additionally a marker, such as a transduction marker (preferably a truncated epidermal growth factor receptor; EGFRt), so that a larger number of CAAR-positive T cells can be enriched. As a further advantage, constructs with additional transduction markers may enable, in an in vivo setting, controlled ending of the therapy through treatment with a therapeutic antibody such as cetuximab, as rescue medication. These constructs therefore comprise transgene-encoded cell surface polypeptides for selection, in vivo tracking and/or ablation of engineered cells.

As is demonstrated in the examples below, the transmembrane, costimulatory and signaling domains as described herein, optionally in combination with the linkers described herein, lead to effective autoantibody-specific B cell depletion. These preferred embodiments are nonlimiting and a skilled person is capable of employing alternative CAR constructs in place of those preferred embodiments mentioned herein.

In some embodiments, the CAAR of the present invention is characterized in that the co-stimulatory domain (transmembrane and intracellular signaling domain) comprises a signaling domain from any one or more of CD28, CD137 (4-1 BB), ICOS, CD134 (OX40), DapIO, CD27, CD2, CD5, ICAM-1, LFA-1, Lck, TNFR-J, TNFR-II, Fas, CD30, CD40 and combinations thereof.

In some embodiments, the CAAR of the present invention is characterized in that the transmembrane domain is selected from an artificial hydrophobic sequence and transmembrane domains of a Type I transmembrane protein, an alpha, beta or zeta chain of a T cell receptor, CD28, ICOS, CD3 epsilon, CD45, CD4, CD5, CD8, CD9, CD16, CD22, CD33, CD37, CD64, CD80, CD86, CD134, CD137, and CD154.

In some embodiments, the CAAR of the present invention is characterized in that the intracellular signaling domain comprises a signaling domain of one or more of a human CD3 zeta chain, FcyRIII, FccRI, a cytoplasmic tail of a Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d, and combinations thereof.

The embodiments described below represent preferred but non-limiting embodiments of the CAAR constructs. Variation in the particular domains described below is contemplated and encompassed by the invention.

In one embodiment, the CAAR-encoding nucleic acid molecule as described herein is characterized in that:
the transmembrane domain is a CD8 alpha, CD28 or ICOS transmembrane domain;
the intracellular domain comprises a CD137 (4-1BB), CD28 or ICOS co-stimulatory domain;
the intracellular domain comprises a CD3 zeta chain signaling domain; and/or
the nucleic acid molecule comprises additionally encodes one or more leader, linker and/or spacer polypeptides positioned N-terminally of the extracellular domain and/or between the extracellular domain and transmembrane domain and/or between the transmembrane domain and intracellular domain.

In one embodiment, the CAAR-encoding nucleic acid molecule as described herein, is characterized in that the nucleic acid molecule encodes:
i. an extracellular domain comprising an autoantigen, comprising or consisting of a nicotinic acetylcholine receptor (nAChR) alpha-1 subunit isoform 1 (SEQ ID NO: 1), alpha-1 subunit isoform 2 (SEQ ID NO: 2), beta-1 subunit isoform 1 (SEQ ID NO: 3), beta-1 subunit isoform 2 (SEQ ID NO: 4), gamma subunit isoform 1 (SEQ ID NO: 5), gamma subunit isoform 2 (SEQ ID NO: 6), delta subunit isoform 1 (SEQ ID NO: 7), delta subunit isoform 2 (SEQ ID NO: 8), epsilon subunit (SEQ ID NO: 9), or an autoantigenic fragment and/or combination and/or variant with at least 80% sequence identity thereto, optionally comprising a linker,
   preferably comprising or consisting of an extracellular autoantigenic part of an alpha-1 subunit isoform 1 (SEQ ID NO: 10), a combination of extracellular autoantigenic parts of alpha-1 isoform 1 and beta-1 isoform 1 subunits (SEQ ID NO: 11) or an extracellular autoantigenic part of a gamma subunit isoform 1 (SEQ ID NO: 12) of a nicotinic acetylcholine receptor (nAChR), or variant with at least 80% sequence identity thereto;
ii. optionally a linker polypeptide positioned between the extracellular domain and transmembrane domain, preferably comprising a sequence according to SEQ ID NO 13, or a sequence with at least 80% sequence identity thereto;
iii. a CD8 alpha transmembrane domain, preferably comprising a sequence according to SEQ ID NO 15, or a sequence with at least 80% sequence identity thereto; and
iv. an intracellular signaling domain comprising a CD137 (4-1BB) co-stimulatory domain and a CD3 zeta chain signaling domain, preferably comprising a sequence according to SEQ ID NO 16 (CD137) and SEQ ID NO 17 (CD3z), or sequences with at least 80% sequence identity thereto, wherein optionally a linker sequence is positioned between the co-stimulatory and signaling domains.

In one embodiment, as an example of the embodiment above, the CAAR-encoding nucleic acid molecule as described herein, is characterized in that the nucleic acid molecule comprises a sequence that encodes:
i. an extracellular domain comprising an autoantigen, comprising or consisting of a nicotinic acetylcholine receptor (nAChR) beta-1 subunit isoform 1 (SEQ ID NO: 3), beta-1 subunit isoform 2 (SEQ ID NO: 4), or the ECD of beta-1 subunit isoform 1 (SEQ ID NO: 21), or an autoantigenic fragment and/or combination and/or variant with at least 80% sequence identity thereto, optionally comprising a linker, or
   preferably an extracellular domain comprising an autoantigen, comprising or consisting of a nicotinic acetylcholine receptor (nAChR) beta-1 subunit isoform 1 (SEQ ID NO: 3) or an autoantigenic fragment and/or variant with at least 80% sequence identity thereto, optionally comprising a linker, or
   preferably an extracellular domain comprising an autoantigen, comprising or consisting of a nicotinic acetylcholine receptor (nAChR) ECD of beta-1 subunit isoform 1 (SEQ ID NO: 21), or an autoantigenic fragment and/or variant with at least 80% sequence identity thereto, optionally comprising a linker.

In one embodiment, as an example of the embodiment above, the CAAR-encoding nucleic acid molecule as described herein, is characterized in that the nucleic acid molecule comprises a sequence that encodes:
i. an extracellular domain comprising an autoantigen, comprising or consisting of a nicotinic acetylcholine receptor (nAChR) alpha-1 subunit isoform 1 (SEQ ID NO: 1), or an autoantigenic fragment and/or variant with at least 80% sequence identity thereto, optionally comprising a linker.

In one embodiment, the invention relates to a chimeric autoantibody receptor (CAAR) polypeptide, comprising:
i. an extracellular domain comprising an autoantigen, comprising or consisting of a nicotinic acetylcholine receptor (nAChR) alpha-1 subunit isoform 1 (SEQ ID NO: 1), alpha-1 subunit isoform 2 (SEQ ID NO: 2), beta-1 subunit isoform 1 (SEQ ID NO: 3), beta-1 subunit isoform 2 (SEQ ID NO: 4), gamma subunit isoform 1 (SEQ ID NO: 5), gamma subunit isoform 2 (SEQ ID NO: 6), delta subunit isoform 1 (SEQ ID NO: 7), delta subunit isoform 2 (SEQ ID NO: 8), epsilon subunit (SEQ ID NO: 9), or an autoantigenic fragment and/or combination and/or variant with at least 80% sequence identity thereto, optionally comprising a linker,
   preferably comprising or consisting of an extracellular autoantigenic part of an alpha-1 subunit isoform 1 (SEQ ID NO: 10), a combination of extracellular autoantigenic parts of alpha-1 isoform 1 and beta-1 isoform 1 subunits (SEQ ID NO: 11) or an extracellular autoantigenic part of a gamma subunit isoform 1 (SEQ ID NO: 12) of a nicotinic acetylcholine receptor (nAChR), or variant with at least 80% sequence identity thereto;
ii. optionally a linker polypeptide positioned between the extracellular domain and transmembrane domain, preferably comprising a sequence according to SEQ ID NO 13, or a sequence with at least 80% sequence identity thereto;
iii. a CD8 alpha transmembrane domain, preferably comprising a sequence according to SEQ ID NO 15, or a sequence with at least 80% sequence identity thereto; and
iv. an intracellular signaling domain comprising a CD137 (4-1BB) co-stimulatory domain and a CD3 zeta chain signaling domain, preferably comprising a sequence according to SEQ ID NO 16 (CD137) and SEQ ID NO 17 (CD3z), or sequences with at least 80% sequence identity thereto, wherein optionally a linker sequence is positioned between the co-stimulatory and signaling domains.

In one embodiment, as an example of the embodiment above, the chimeric autoantibody receptor (CAAR) polypeptide comprises:
i. an extracellular domain comprising an autoantigen, comprising or consisting of a nicotinic acetylcholine receptor (nAChR) beta-1 subunit isoform 1 (SEQ ID NO: 3), beta-1 subunit isoform 2 (SEQ ID NO: 4), or the ECD of beta-1 subunit isoform 1 (SEQ ID NO: 21), or an autoantigenic fragment and/or combination and/or variant with at least 80% sequence identity thereto, optionally comprising a linker
   preferably an extracellular domain comprising an autoantigen, comprising or consisting of a nicotinic acetylcholine receptor (nAChR) beta-1 subunit isoform 1 (SEQ ID NO: 3) or an autoantigenic fragment and/or variant with at least 80% sequence identity thereto, optionally comprising a linker, or
   preferably an extracellular domain comprising an autoantigen, comprising or consisting of a nicotinic acetylcholine receptor (nAChR) ECD of beta-1 subunit isoform 1 (SEQ ID NO: 21), or an autoantigenic fragment and/or variant with at least 80% sequence identity thereto, optionally comprising a linker.

In one embodiment, as an example of the embodiment above, the chimeric autoantibody receptor (CAAR) polypeptide comprises:
i. an extracellular domain comprising an autoantigen, comprising or consisting of a nicotinic acetylcholine receptor (nAChR) alpha-1 subunit isoform 1 (SEQ ID NO: 1), or an autoantigenic fragment and/or variant with at least 80% sequence identity thereto, optionally comprising a linker.

In one embodiment, the invention relates to an isolated nucleic acid molecule, optionally in the form of an isolated vector, such as an isolated viral vector or transposon, selected from the group consisting of:
a) a nucleic acid molecule comprising a nucleotide sequence
   which encodes a CAAR polypeptide as described herein,
   which encodes a targeting (i.e. an extracellular auto-antibody-binding) domain or part thereof, the sequence comprising one or more of SEQ ID NOs 1 to 12 or 21, and/or
   which encodes a CAAR polypeptide as described herein, the sequence comprising one or more of SEQ ID NOs 18 to 20 or 22;
b) a nucleic acid molecule which is complementary to a nucleotide sequence in accordance with a);
c) a nucleic acid molecule comprising a nucleotide sequence having sufficient sequence identity to be functionally analogous/equivalent to a nucleotide sequence according to a) or b), comprising preferably a sequence identity to a nucleotide sequence according to a) or b) of at least 50%, preferably 60%, 70%, 80%, 85%, 90%, or 95%;
d) a nucleic acid molecule which, as a consequence of the genetic code, is degenerated into a nucleotide sequence according to a) through c); and/or
e) a nucleic acid molecule according to a nucleotide sequence of a) through d) which is modified by deletions, additions, substitutions, translocations, inversions and/or insertions and is functionally analogous/equivalent to a nucleotide sequence according to a) through d).

Variation in length of the nucleotide or amino acid sequences as described herein is also encompassed by the present invention. A skilled person is capable of providing nucleic acid sequence variants that are longer or shorter than the specific coding sequences described herein, which will still exhibit sufficient similarity to code for the proteins described herein in order to provide the outcomes desired.

For example, shorter variants of SEQ ID NO 1 to 12 or 21, which represent the autoantigens of the invention, comprising 1, 2, 3, 4, 5, 6, 7, 8, 9, 10, 20, 30, 40, or up to 50 amino acids less than the disclosed form may also enable effective autoantigen properties. Fragments of SEQ ID NO 1 to 12 or 21 are therefore also considered. Additionally, longer variants of SEQ ID NO 1 to 12 or 21 comprising 1, 2, 3, 4, 5, 6, 7, 8, 9, 10, 20, 30, 40, or up to 50 amino acids of any given additional sequence more than SEQ ID NO 1 to 12 or 21 may also enable effective outcomes. The amino acid sequences may comprise 0 to 100, 2 to 50, 5 to 20, or for example 8 to 15, or any value from 0 to 20, amino acid additions or deletions at either the N- and/or C-terminus of the proteins of SEQ ID NO 1 to 12 or 21. The termini may also be modified with additional linker sequences, or removal of sequences, as long as the properties of the protein with respect to autoantibody binding are essentially maintained.

In other embodiments of the invention, the autoantigen protein employed may comprise or consist of an amino acid sequence with at least 50%, 60%, 70%, 80%, 90% or 95% sequence identity to SEQ ID NO 1 to 12 or 21. Preferably the sequence variant comprises at least 80%, 90%, 91, 92, 93, 94, 95, 96, 97, 98 or 99% sequence identity to SEQ ID NO 1 to 12 or 21 and preferably exhibits functional analogy to the specific human proteins described herein. Functional analogy is assessed via determining the same or a similar autoantigen-antibody binding and/or autoantibody-specific B cell depletion as described herein. Suitable in vitro assays for determining the desired binding are known to a skilled person.

In one embodiment, the invention relates to a CAAR according to a sequence of SEQ ID NO 22, 18, 19 or 20, or a variant with at least 80% sequence identity thereto, or to a nucleic acid molecule encoding said CAAR.

In a further aspect, the invention relates to a vector comprising a nucleic acid molecule encoding a chimeric autoantibody receptor (CAAR) as described herein.

In some embodiments, the vector is a viral vector, such as a lentiviral vector or retroviral vector.

In some embodiments, the vector is a nanoparticle as a transfection vehicle.

In some embodiments, the vector is a transposon or an RNA vector.

In some embodiments, the vector is a sleeping beauty transposon, preferably a SB100/pT4 sleeping beauty transposon.

In some embodiments, the vector is suitable for integration of the CAAR encoding sequence into a cell via CRISPR/Cas9-mediated gene modification.

In order to express a desired polypeptide, a nucleotide sequence encoding the CAAR polypeptide, can be inserted into appropriate vector. Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or MI 3 phage, and animal viruses. CAAR-encoding nucleotide sequences may also be present in the form suitable for integration into a cell via CRISPR/Cas9-mediated gene modification.

An additional and surprising aspect of the invention is an improved stability of the CAAR as disclosed herein. The CAAR polypeptide can readily be stored for extended periods under appropriate conditions without any loss of binding affinity.

Preferred amino acid and nucleotide sequences of the present invention:

SEQ ID NO: 1
CHRNA1-Isoform 1
MEPWPLLLLFSLCSAGLVLGSEHETRLVAKLFKDYSSVVRPVEDHRQVVEVTVGLQLIQLINVDEVNQI
VTTNVRLKQQWVDYNLKWNPDDYGGVKKIHIPSEKIWRPDLVLYNNADGDFAIVKFTKVLLQYTGHIT
WTPPAIFKSYCEIIVTHFPFDEQNCSMKLGTWTYDGSVVAINPESDQPDLSNFMESGEWVIKESRGWK
HSVTYSCCPDTPYLDITYHFVMQRLPLYFIVNVIIPCLLFSFLTGLVFYLPTDSGEKMTLSISVLLSLTVFL
LVIVELIPSTSSAVPLIGKYMLFTMVFVIASIIITVIVINTHHRSPSTHVMPNWVRKVFIDTIPNIMFFSTMKR
PSREKQDKKIFTEDIDISDISGKPGPPPMGFHSPLIKHPEVKSAIEGIKYIAETMKSDQESNNAAAEWKY
VAMVMDHILLGVFMLVCIIGTLAVFAGRLIELNQQG
SEQ ID NO: 2
CHRNA1-Isoform 2
MEPWPLLLLFSLCSAGLVLGSEHETRLVAKLFKDYSSVVRPVEDHRQVVEVTVGLQLIQLINVDEVNQI
VTTNVRLKQGDMVDLPRPSCVTLGVPLFSHLQNEQWVDYNLKWNPDDYGGVKKIHIPSEKIWRPDLV
LYNNADGDFAIVKFTKVLLQYTGHITWTPPAIFKSYCEIIVTHFPFDEQNCSMKLGTWTYDGSVVAINPE
SDQPDLSNFMESGEWVIKESRGWKHSVTYSCCPDTPYLDITYHFVMQRLPLYFIVNVIIPCLLFSFLTGL
VFYLPTDSGEKMTLSISVLLSLTVFLLVIVELIPSTSSAVPLIGKYMLFTMVFVIASIIITVIVINTHHRSPSTH
VMPNWVRKVFIDTIPNIMFFSTMKRPSREKQDKKIFTEDIDISDISGKPGPPPMGFHSPLIKHPEVKSAIE
GIKYIAETMKSDQESNNAAAEWKYVAMVMDHILLGVFMLVCIIGTLAVFAGRLIELNQQG
SEQ ID NO: 3
CHRNB1-Isoform 1
MTPGALLMLLGALGAPLAPGVRGSEAEGRLREKLFSGYDSSVRPAREVGDRVRVSVGLILAQLISLNE
KDEEMSTKVYLDLEWTDYRLSWDPAEHDGIDSLRITAESVWLPDVVLLNNNDGNFDVALDISVVVSSD
GSVRWQPPGIYRSSCSIQVTYFPFDWQNCTMVFSSYSYDSSEVSLQTGLGPDGQGHQEIHIHEGTFIE
NGQWEIIHKPSRLIQPPGDPRGGREGQRQEVIFYLIIRRKPLFYLVNVIAPCILITLLAIFVFYLPPDAGEK
MGLSIFALLTLTVFLLLLADKVPETSLSVPIIIKYLMFTMVLVTFSVILSVVVLNLHHRSPHTHQMPLWVRQ
IFIHKLPLYLRLKRPKPERDLMPEPPHCSSPGSGWGRGTDEYFIRKPPSDFLFPKPNRFQPELSAPDLR
RFIDGPNRAVALLPELREVVSSISYIARQLQEQEDHDALKEDWQFVAMVVDRLFLWTFIIFTSVGTLVIFL
DATYHLPPPDPFP
SEQ ID NO: 4
CHRNB1-Isoform 2
MSTKVYLDLEWTDYRLSWDPAEHDGIDSLRITAESVWLPDVVLLNNNDGNFDVALDISVVVSSDGSVR
WQPPGIYRSSCSIQVTYFPFDWQNCTMVFSSYSYDSSEVSLQTGLGPDGQGHQEIHIHEGTFIENGQ
WEIIHKPSRLIQPPGDPRGGREGQRQEVIFYLIIRRKPLFYLVNVIAPCILITLLAIFVFYLPPDAGEKMGLS
IFALLTLTVFLLLLADKVPETSLSVPIIIKYLMFTMVLVTFSVILSVVVLNLHHRSPHTHQMPLWVRQIFIHK
LPLYLRLKRPKPERDLMPEPPHCSSPGSGWGRGTDEYFIRKPPSDFLFPKPNRFQPELSAPDLRRFID
GPNRAVALLPELREVVSSISYIARQLQEQEDHDALKEDWQFVAMVVDRLFLWTFIIFTSVGTLVIFLDAT
YHLPPPDPFP
SEQ ID NO: 5
CHRNG-Isoform 1
MHGGQGPLLLLLLLAVCLGAQGRNQEERLLADLMQNYDPNLRPAERDSDVVNVSLKLTLTNLISLNER
EEALTTNVWIEMQWCDYRLRWDPRDYEGLWVLRVPSTMVWRPDIVLENNVDGVFEVALYCNVLVSP
DGCIYWLPPAIFRSACSISVTYFPFDWQNCSLIFQSQTYSTNEIDLQLSQEDGQTIEWIFIDPEAFTENG
EWAIQHRPAKMLLDPAAPAQEAGHQKVVFYLLIQRKPLFYVINIIAPCVLISSVAILIHFLPAKAGGQKCTV
AINVLLAQTVFLFLVAKKVPETSQAVPLISKYLTFLLVVTILIVVNAVVVLNVSLRSPHTHSMARGVRKVFL
RLLPQLLRMHVRPLAPAAVQDTQSRLQNGSSGWSITTGEEVALCLPRSELLFQQWQRQGLVAAALEK
LEKGPELGLSQFCGSLKQAAPAIQACVEACNLIACARHQQSHFDNGNEEWFLVGRVLDRVCFLAMLSL
FICGTAGIFLMAHYNRVPALPFPGDPRPYLPSPD
SEQ ID NO: 6
CHRNG-Isoform 2
MHGGQGPLLLLLLLAVCLGAQGRNQEERLLADLMQNYDPNLRPAERDSDVVNVSLKLTLTNLISLNER
EEALTTNVWIEMQWCDYRLRWDPRDYEGLWVLRVPSTMVWRPDIVLENKSQTYSTNEIDLQLSQED
GQTIEWIFIDPEAFTENGEWAIQHRPAKMLLDPAAPAQEAGHQKVVFYLLIQRKPLFYVINIIAPCVLISSV
AILIHFLPAKAGGQKCTVAINVLLAQTVFLFLVAKKVPETSQAVPLISKYLTFLLVVTILIVVNAVVVLNVSL
RSPHTHSMARGVRKVFLRLLPQLLRMHVRPLAPAAVQDTQSRLQNGSSGWSITTGEEVALCLPRSEL
LFQQWQRQGLVAAALEKLEKGPELGLSQFCGSLKQAAPAIQACVEACNLIACARHQQSHFDNGNEEW
FLVGRVLDRVCFLAMLSLFICGTAGIFLMAHYNRVPALPFPGDPRPYLPSPD
SEQ ID NO: 7
CHRND-Isoform 1
MEGPVLTLGLLAALAVCGSWGLNEEERLIRHLFQEKGYNKELRPVAHKEESVDVALALTLSNLISLKEV
EETLTTNVWIEHGWTDNRLKWNAEEFGNISVLRLPPDMVWLPEIVLENNNDGSFQISYSCNVLVYHYG
FVYWLPPAIFRSSCPISVTYFPFDWQNCSLKFSSLKYTAKEITLSLKQDAKENRTYPVEWIIIDPEGFTEN
GEWEIVHRPARVNVDPRAPLDSPSRQDITFYLIIRRKPLFYIINILVPCVLISFMVNLVFYLPADSGEKTSV
AISVLLAQSVFLLLISKRLPATSMAIPLIGKFLLFGMVLVTMVVVICVIVLNIHFRTPSTHVLSEGVKKLFLE
TLPELLHMSRPAEDGPSPGALVRRSSSLGYISKAEEYFLLKSRSDLMFEKQSERHGLARRLTTARRPP
ASSEQAQQELFNELKPAVDGANFIVNHMRDQNNYNEEKDSWNRVARTVDRLCLFVVTPVMVVGTAWI
FLQGVYNQPPPQPFPGDPYSYNVQDKRFI
SEQ ID NO: 8
CHRND-Isoform 2
MEGPVLTLGLLAALAVCGSWGLNEEERLIRHLFQEKGYNKELRPVAHKEESVDVALALTLSNLISLGWT
DNRLKWNAEEFGNISVLRLPPDMVWLPEIVLENNNDGSFQISYSCNVLVYHYGFVYWLPPAIFRSSCPI
SVTYFPFDWQNCSLKFSSLKYTAKEITLSLKQDAKENRTYPVEWIIIDPEGFTENGEWEIVHRPARVNV
DPRAPLDSPSRQDITFYLIIRRKPLFYIINILVPCVLISFMVNLVFYLPADSGEKTSVAISVLLAQSVFLLLIS
KRLPATSMAIPLIGKFLLFGMVLVTMVVVICVIVLNIHFRTPSTHVLSEGVKKLFLETLPELLHMSRPAED
GPSPGALVRRSSSLGYISKAEEYFLLKSRSDLMFEKQSERHGLARRLTTARRPPASSEQAQQELFNEL
KPAVDGANFIVNHMRDQNNYNEEKDSWNRVARTVDRLCLFVVTPVMVVGTAWIFLQGVYNQPPPQP
FPGDPYSYNVQDKRFI
SEQ ID NO: 9
CHRNE
MARAPLGVLLLLGLLGRGVGKNEELRLYHHLFNNYDPGSRPVREPEDTVTISLKVTLTNLISLNEKEETL
TTSVWIGIDWQDYRLNYSKDDFGGIETLRVPSELVWLPEIVLENNIDGQFGVAYDANVLVYEGGSVTW
LPPAIYRSVCAVEVTYFPFDWQNCSLIFRSQTYNAEEVEFTFAVDNDGKTINKIDIDTEAYTENGEWAID
FCPGVIRRHHGGATDGPGETDVIYSLIIRRKPLFYVINIIVPCVLISGLVLLAYFLPAQAGGQKCTVSINVL
LAQTVFLFLIAQKIPETSLSVPLLGRFLIFVMVVATLIVMNCVIVLNVSQRTPTTHAMSPRLRHVLLELLPR
LLGSPPPPEAPRAASPPRRASSVGLLLRAEELILKKPRSELVFEGQRHRQGTWTAAFCQSLGAAAPEV
RCCVDAVNFVAESTRDQEATGEEVSDWVRMGNALDNICFWAALVLFSVGSSLIFLGAYFNRVPDLPY
APCIQP
SEQ ID NO: 10
ECD of CHRNA1 Isoform 1
MEPWPLLLLFSLCSAGLVLGSEHETRLVAKLFKDYSSVVRPVEDHRQVVEVTVGLQLIQLINVDEVNQI
VTTNVRLKQQWVDYNLKWNPDDYGGVKKIHIPSEKIWRPDLVLYNNADGDFAIVKFTKVLLQYTGHIT
WTPPAIFKSYCEIIVTHFPFDEQNCSMKLGTWTYDGSVVAINPESDQPDLSNFMESGEWVIKESRGWK
HSVTYSCCPDTPYLDITYHFVMQRLP
SEQ ID NO: 11
ECD of CHNRA1 Isoform 1 and ECD of CHRNB1 Isoform1
MEPWPLLLLFSLCSAGLVLGSEHETRLVAKLFKDYSSVVRPVEDHRQVVEVTVGLQLIQLINVDEVNQI
VTTNVRLKQQWVDYNLKWNPDDYGGVKKIHIPSEKIWRPDLVLYNNADGDFAIVKFTKVLLQYTGHIT
WTPPAIFKSYCEIIVTHFPFDEQNCSMKLGTWTYDGSVVAINPESDQPDLSNFMESGEWVIKESRGWK
HSVTYSCCPDTPYLDITYHFVMQRLAGSAGSAGSAGSAGSAGSAGSAGSSEAEGRLREKLFSGYDSS
VRPAREVGDRVRVSVGLILAQLISLNEKDEEMSTKVYLDLEWTDYRLSWDPAEHDGIDSLRITAESVWL
PDVVLLNNNDGNFDVALDISVVVSSDGSVRWQPPGIYRSSCSIQVTYFPFDWQNCTMVFSSYSYDSS
EVSLQTGLGPDGQGHQEIHIHEGTFIENGQWENIHKPSRLIQPPGDPRGGREGQRQEVIFYLIIRRK
SEQ ID NO: 12
ECD of CHRNG Isoform 1
MHGGQGPLLLLLLLAVCLGAQGRNQEERLLADLMQNYDPNLRPAERDSDVVNVSLKLTLTNLISLNER
EEALTTNVWIEMQWCDYRLRWDPRDYEGLWVLRVPSTMVWRPDIVLENNVDGVFEVALYCNVLVSP
DGCIYWLPPAIFRSACSISVTYFPFDWQNCSLIFQSQTYSTNEIDLQLSQEDGQTIEWIFIDPEAFTENG
EWAIQHRPAKMLLDPAAPAQEAGHQKVVFYLLIQRK
SEQ ID NO: 13
Linker-1
ASGGGGSGGGGSSG
SEQ ID NO: 14
alpha-beta-Linker-2
AGSAGSAGSAGSAGSAGSAGSAGS
SEQ ID NO: 15
CD8 transmembrane region
IYIWAPLAGTCGVLLLSLVITLYC
SEQ ID NO: 16
CD137 co-stimulatory domain
KRGRKKLLYIFKQPFMRPVQTTQEEDGCSCRFPEEEEGGCEL
SEQ ID NO: 17
CD3z activation domain
RVKFSRSADAPAYQQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
SEQ ID NO: 18
AChRa1-CAAR; ECD of CHRNA1 Isoform 1
MEPWPLLLLFSLCSAGLVLGSEHETRLVAKLFKDYSSVVRPVEDHRQVVEVTVGLQLIQLINVDEVNQI
VTTNVRLKQQWVDYNLKWNPDDYGGVKKIHIPSEKIWRPDLVLYNNADGDFAIVKFTKVLLQYTGHIT
WTPPAIFKSYCEIIVTHFPFDEQNCSMKLGTWTYDGSVVAINPESDQPDLSNFMESGEWVIKESRGWK
HSVTYSCCPDTPYLDITYHFVMQRLPASGGGGSGGGGSSGIYIWAPLAGTCGVLLLSLVITLYCKRGR
KKLLYIFKQPFMRPVQTTQEEDGCSCRFPEEEEGGCELRVKFSRSADAPAYQQGQNQLYNELNLGRR
EEYDVLDKRRGRDPEMGGKPRRKNPQEGLYNELQKDKMAEAYSEIGMKGERRRGKGHDGLYQGLS
TATKDTYDALHMQALPPR
SEQ ID NO: 19
ACHRa1-b1-CAAR; ECD of CHNRA1 Isoform 1 and ECD of CHRNB1 Isoform1
MEPWPLLLLFSLCSAGLVLGSEHETRLVAKLFKDYSSVVRPVEDHRQVVEVTVGLQLIQLINVDEVNQI
VTTNVRLKQQWVDYNLKWNPDDYGGVKKIHIPSEKIWRPDLVLYNNADGDFAIVKFTKVLLQYTGHIT
WTPPAIFKSYCEIIVTHFPFDEQNCSMKLGTWTYDGSVVAINPESDQPDLSNFMESGEWVIKESRGWK
HSVTYSCCPDTPYLDITYHFVMQRLAGSAGSAGSAGSAGSAGSAGSAGSSEAEGRLREKLFSGYDSS
VRPAREVGDRVRVSVGLILAQLISLNEKDEEMSTKVYLDLEWTDYRLSWDPAEHDGIDSLRITAESVWL
PDVVLLNNNDGNFDVALDISVVVSSDGSVRWQPPGIYRSSCSIQVTYFPFDWQNCTMVFSSYSYDSS
EVSLQTGLGPDGQGHQEIHIHEGTFIENGQWENIHKPSRLIQPPGDPRGGREGQRQEVIFYLIIRRKAS
GGGGSGGGGSSGIYIWAPLAGTCGVLLLSLVITLYCKRGRKKLLYIFKQPFMRPVQTTQEEDGCSCRF
PEEEEGGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
SEQ ID NO: 20
AChRg-CAAR; ECD of CHRNG Isoform 1
MHGGQGPLLLLLLLAVCLGAQGRNQEERLLADLMQNYDPNLRPAERDSDVVNVSLKLTLTNLISLNER
EEALTTNVWIEMQWCDYRLRWDPRDYEGLWVLRVPSTMVWRPDIVLENNVDGVFEVALYCNVLVSP
DGCIYWLPPAIFRSACSISVTYFPFDWQNCSLIFQSQTYSTNEIDLQLSQEDGQTIEWIFIDPEAFTENG
EWAIQHRPAKMLLDPAAPAQEAGHQKVVFYLLIQRKASGGGGSGGGGSSGIYIWAPLAGTCGVLLLSL
VITLYCKRGRKKLLYIFKQPFMRPVQTTQEEDGCSCRFPEEEEGGCELRVKFSRSADAPAYQQGQNQ
LYNELNLGRREEYDVLDKRRGRDPEMGGKPRRKNPQEGLYNELQKDKMAEAYSEIGMKGERRRGK
GHDGLYQGLSTATKDTYDALHMQALPPR
SEQ ID NO: 21
ECD of CHRNB1 (first 244 amino acids of SEQ ID NO 3)
MTPGALLMLLGALGPPLAPGVRGSEAEGRLREKLFSGYDSSVRPAREVGDRVRVSVGLILAQLISLNE
KDEEMSTKVYLDLEWTDYRLSWDPAEHDGIDSLRITAESVWLPDVVLLNNNDGNFDVALDISVVVSSD
GSVRWQPPGIYRSSCSIQVTYFPFDWQNCTMVFSSYSYDSSEVSLQTGLGPDGQGHQEIHIHEGTFIE
NGQWENIHKPSRLIQPPGDPRGGREGQRQEVIFYLIIRRK
SEQ ID NO 22:
ACHRb1-CAAR; ECD of CHNRB1 Isoform 1; Full construct (ECD of CHRNB1
(SEQ ID NO 21) + SEQ ID NO 13 + SEQ ID NO 15 + SEQ ID NO 16 + SEQ ID NO 17)
MTPGALLMLLGALGPPLAPGVRGSEAEGRLREKLFSGYDSSVRPAREVGDRVRVSVGLILAQLISLNE
KDEEMSTKVYLDLEWTDYRLSWDPAEHDGIDSLRITAESVWLPDVVLLNNNDGNFDVALDISVVVSSD
GSVRWQPPGIYRSSCSIQVTYFPFDWQNCTMVFSSYSYDSSEVSLQTGLGPDGQGHQEIHIHEGTFIE
NGQWENIHKPSRLIQPPGDPRGGREGQRQEVIFYLIIRRKASGGGGSGGGGSSGIYIWAPLAGTCGVL
LLSLVITLYCKRGRKKLLYIFKQPFMRPVQTTQEEDGCSCRFPEEEEGGCELRVKFSRSADAPAYQQG
QNQLYNELNLGRREEYDVLDKRRGRDPEMGGKPRRKNPQEGLYNELQKDKMAEAYSEIGMKGERR
RGKGHDGLYQGLSTATKDTYDALHMQALPPR

In a further aspect, the invention relates to a genetically modified immune cell comprising a nucleic acid molecule encoding a CAAR as described herein, or a vector comprising such a nucleic acid molecule and/or expressing a CAAR as described herein.

In one embodiment, the genetically modified immune cell is in combination with other genetically modified immune cells of the invention.

In embodiments, a genetically modified immune cell comprising an inventive CAAR with a beta-1 subunit autoantigen is in combination with a genetically modified immune cell comprising an inventive CAAR with an alpha-1 subunit autoantigen.

In embodiments, a genetically modified immune cell comprising an inventive CAAR with a beta-1 subunit autoantigen, e.g. comprising or consisting of a nicotinic acetylcholine receptor (nAChR) beta-1 subunit isoform 1 (SEQ ID NO: 3), beta-1 subunit isoform 2 (SEQ ID NO: 4), or the ECD of beta-1 subunit isoform 1 (SEQ ID NO: 21), or an autoantigenic fragment and/or combination and/or variant thereof, is in combination with a genetically modified immune cell comprising an inventive CAAR with an alpha-1 subunit autoantigen, e.g. alpha-1 subunit isoform 1 (SEQ ID NO: 1), alpha-1 subunit isoform 2 (SEQ ID NO: 2), extracellular autoantigenic part of an alpha-1 subunit isoform 1 (SEQ ID NO: 10), or a combination of extracellular autoantigenic parts of alpha-1 isoform 1 and beta-1 isoform 1 subunits (SEQ ID NO: 11).

In one embodiment, the genetically modified immune cell is selected from the group consisting of a T cell, an NK cell, a macrophage or a dendritic cell.

In one embodiment, the genetically modified immune cell as described herein is a T lymphocyte (T cell) and said T lymphocyte is a CD8+ and/or CD4+ cytotoxic T lymphocyte, or mixture thereof.

In some embodiments, CAAR-engineered immune cells can be edited for deletion of TCRs to avoid GVHD reactions. In some embodiments, CAAR-engineered immune cells can be edited for deletion of HLA to avoid allogeneic rejection and become “universal CAAR-T cells”.

In some embodiments the immune cell is preferably a T lymphocyte, an NK cell, a macrophage or a dendritic cell. In some preferred embodiments, the immune cell is cytotoxic, preferably cytotoxic towards autoantibody-presenting and/or secreting B cells. Cytotoxic immune cells are known in the field to exhibit cytolytic and/or other beneficial activity in response to unwanted agents, cells or pathogens. By directing the activity of these cells to particular immunogenic targets, namely the autoantigens described herein, pathogenic cells can be eliminated by the corresponding activity of the immune cell described herein.

In a preferred embodiment, the immune cell is a T lymphocyte, preferably a cytotoxic T lymphocyte or a T helper cell.

In some embodiments, the CAAR-engineered immune cell could be engineered to additionally co-express cytokines (such as IL-15, IL-12, IFN-gamma, IFN-alpha, GM-CSF, FLT3L, IL-21, IL-23) or co-stimulatory ligands (CD80, CD86, CD40L) to improve the immune therapeutic effects.

In some embodiments, the CAAR-engineered immune cell could be engineered to additionally co-express siRNAs or shRNAs or miRNAs to down-regulate, or could be genetically edited with CRISPR/Cas, to knock-out expression of the T cell receptor and the major histocompatibility complex, such that these cells can be used as allogeneic cell therapies.

In some embodiments, the CAAR-engineered immune cell could be engineered to additionally co-express siRNAs or shRNAs or miRNAs to down-regulate, or could be genetically edited with the CRISPR/Cas, to knock-out expression of check point molecules on the T cell surface (PD1, Tim3, LAG, etc. . . . ).

Combined approaches employing down-regulation of the major histocompatibility complex or check point molecules on the T cell surface lead to additional, potentially synergistic effects, in optimizing the local immune environment to enhance the cytolytic effect of the CAAR-engineered immune cells of the invention against the pathogenic B cells.

In a further aspect, the invention relates to an immune cell as described herein for use in the treatment and/or prevention of a neuromuscular disorder associated with autoantibodies that bind a nicotinic acetylcholine receptor (nAChR).

In one embodiment, the invention relates to an immune cell as described herein for use in the treatment and/or prevention of myasthenia gravis (MG).

In one embodiment, the invention relates to an immune cell as described herein for use in the treatment and/or prevention of arthrogryposis multiplex congenita (AMC) caused by diaplacental transfer of autoantibodies.

The invention therefore relates to the medical use of the CAAR-engineered immune cells. The invention therefore also encompasses methods for treating and/or preventing a medical condition as described herein, comprising the administration of an immune cell as described herein (comprising/expressing a CAAR of the present invention) to a subject in need thereof.

According to the invention, the embodiments of any given aspect are considered to apply to other aspects and embodiments, such that combinations of particular embodiments as disclosed herein are contemplated. For example, embodiments disclosed with respect to the medical treatment may be incorporated as functional features of the CAARs, and vice versa.

### DETAILED DESCRIPTION OF THE INVENTION

All cited documents of the patent and non-patent literature are hereby incorporated by reference in their entirety.

### Autoantigen and Disease Description:

The invention relates to a chimeric autoantibody receptor (CAAR) that enables targeting of an immune cell to autoantibody producing B cells, wherein the CAAR comprises an autoantigen or fragment thereof that is bound by autoantibodies associated with autoimmune neuromuscular disorders. Therefore, the invention relates to a chimeric autoantibody receptor (CAAR), wherein the CAR comprises an extracellular domain comprising an autoantigen of a nicotinic acetylcholine receptor (nAChR) or fragment thereof. The autoantigen of the CAAR therefore represents a targeting subunit, equivalent to an extracellular antigen-binding domain of a CAR, that targets the immune cell to the B cell to be depleted.

As used herein, the term “autoantigen or fragment thereof bound by autoantibodies associated with a neuromuscular disorder” represents a functional definition of the autoantigen comprised within the CAAR. A skilled person is capable of determining the autoantigens of this class and the associated medical conditions. Binding between an autoantigen and antibody is, as such, an established phenomenon and reflects essentially the physical interaction between any given antibody and its target.

As used herein, the term “autoimmune neuromuscular disorder” relates to any medical condition with an autoimmune component, in which autoantibodies are present, that lead to neuromuscular disorders. For example, the autoantibodies may affect peripheral nerves, neuromuscular junctions or muscle and can lead to a clinical spectrum with diverse pathogenetic mechanisms. For example, the peripheral nervous system may be targeted by pathogenic mechanisms involving interactions between antigen-presenting cells, B cells and different types of T cells, directed against specific autoantigens predominantly expressed in the peripheral nervous system. Various neurological autoimmune conditions are known to a skilled person, in which the autoantibodies target typically either autoantigens of primarily the central or peripheral nervous system. In preferred embodiments, the medical conditions of the present invention exhibit autoantibodies that target primarily the peripheral nervous system, for example to a greater extent than the central nervous system.

As used herein, the “central nervous system” (CNS) refers to the part of the nervous system consisting of the brain and spinal cord. The CNS is contained within the dorsal body cavity, with the brain housed in the cranial cavity and the spinal cord in the spinal canal. The CNS is divided in white and gray matter.

From and to the spinal cord are projections of the peripheral nervous system in the form of spinal nerves. The nerves connect the spinal cord to skin, joints, muscles etc. and allow for the transmission of efferent motor as well as afferent sensory signals and stimuli. This allows for voluntary and involuntary motions of muscles, as well as the perception of senses.

As used herein, the “peripheral nervous system” (PNS) consists of the nerves and ganglia outside the brain and spinal cord. The main function of the PNS is to connect the CNS to the limbs and organs, essentially serving as a relay between the brain and spinal cord and the rest of the body. Unlike the CNS, the PNS is not protected by the vertebral column and skull, or by the blood-brain barrier.

Included in the PNS is the “neuromuscular junction” (NMJ), a region of synaptic connection between the terminal end of a motor nerve and a muscle (for example a skeletal muscle, smooth muscle, or cardiac muscle). It is the site for the transmission of action potential from nerve to the muscle, and can be the site of disease. For example, diseases of the NMJ produce muscle weakness through different mechanisms that may affect presynaptic, synaptic, or postsynaptic portions of the NMJ. Three main diseases that involve NMJ are Myasthenia Gravis (MG), Lambert-Eaton syndrome (LES), and Botulism.

As used herein, the “autoimmune neuromuscular disorders” are conditions in which the immune systems targets components of the peripheral nerves, neuromuscular junction and/or muscle. Such disorders may have a wide clinical spectrum with diverse pathogenetic mechanisms. Peripheral nervous system may be targeted in the context of complex immune reactions involving different cytokines, antigen-presenting cells, B cells and different types of T cells. Various immunomodulating and cytotoxic treatments block proliferation or activation of immune cells by different mechanisms attempting to control the response of the immune system and limit target organ injury. Most treatment protocols for autoimmune neuromuscular disorders are based on the use of corticosteroids, intravenous immunoglobulins and plasmapheresis, with cytotoxic agents mostly used as steroid-sparing medications.

One example of an autoimmune neuromuscular disorder, and an autoimmune condition primarily targeting the peripheral nervous system, is the condition myasthenia gravis (MG).

Myasthenia gravis is a chronic autoimmune neuromuscular disease that causes weakness in the skeletal muscles, which are responsible for breathing and moving parts of the body, including the arms and legs. Myasthenia gravis is caused by an error in the transmission of nerve impulses to muscles. It occurs when normal communication between the nerve and muscle is interrupted at the neuromuscular junction (NMJ), the place where nerve cells connect with the muscles they control. Autoantibodies target key molecules at the NMJ, such as the nicotinic acetylcholine receptor (AChR), muscle-specific kinase (MuSK), and low-density lipoprotein receptor-related protein 4 (Lrp4), that lead by a range of different pathogenic mechanisms to altered tissue architecture and reduced densities or functionality of AChRs, reduced neuromuscular transmission, and therefore a severe fatigable skeletal muscle weakness.

MG is a disorder with an estimated prevalence of 70-163 per million for acetylcholine receptor (AChR) MG, and around 1.9-2.9 per million for muscle specific kinase (MuSK) MG. Women are more often affected than men, with a female to male ratio of 3:1 for AChR MG and a ratio of 9:1 for MuSK MG. The characterizing symptom is fatigable skeletal muscle weakness. Initial weakness often affects only ocular muscles, manifesting as ptosis (hanging of the eyelid) or diplopia (double vision). Most patients progress to generalized weakness, e.g., of limb muscles, within the first two years after disease onset. Other muscles that can be involved are bulbar muscles, which are necessary for speaking (leading to dysarthria), chewing and swallowing (causing dysphagia). Respiratory muscles can also be affected in up to 20% of cases with AChR MG, leading to a myasthenic crisis where patients need to be ventilated artificially. AChR MG can be further divided into several subgroups: (1) Early-onset MG (EOMG) defines patients with an age of onset below 50 years, and are predominantly females with an onset in the 2nd and 3rd decade, frequently present with thymic hyperplasia; (2) late-onset MG (LOMG) with a higher fraction of male patients, often with an additional presence of striational antibodies; (3) thymoma-associated MG (TAMG), which affects approximately 10% of AChR MG patients; (4) ocular MG (OMG) with predominantly ocular symptoms; and (5) fetal or neonatal forms in which maternal autoantibodies pass the placenta. The passive transfer of antibodies against the adult AChR towards the fetus leads to a mild form of transient MG that passes weeks after birth. The symptoms include hypotonia, impaired sucking, swallowing, and breathing. Patients go into remission after days to months. Antibodies against the fetal form of the AChR cause severe developmental defects and are a cause of arthrogryposis multiplex congenita. For a detailed review refer to Koneczny et al (Cells. 2019 July; 8(7): 671).

The invention therefore relates to a CAAR suitable for treatment of any form of MG, in particular those characterized as AChR MG, in which autoantibodies are present directed against the AChR, and any of the above-mentioned stages or forms of the disease.

Acetylcholine receptors (AChRs) are member of a superfamily of neurotransmitter-gated ion channels, each comprised of five homologous subunits arranged around a central ion channel. AChR subunits are subdivided into four classes. Class I-III represent neuronal AChR subunits and class IV include muscle AChRs. AChR subunits show 35-50% sequence homology in the N-terminal region, are glycosylated, and share structural features. Three highly conserved and mainly α-helical transmembrane domains (M1-M3) encompass between the large extracellular domain and the cytoplasmic domain (containing one α-helix). A fourth α-helical transmembrane domain (M4) crosses back to the extracellular space creating a short (10-20 amino acids) extracellular sequence. The N-terminal extracellular portion is organized around a β-sandwich core and the cytoplasmic domains of AChR β and δ contain a regulated phosphotyrosine site, which is important for cytoskeletal anchorage. Muscle AChRs have the composition α2βδγ in embryonic muscle or α2βδε in adult muscle. ACh binding sites are present at the subunit interfaces, for example at the subunit interfaces αγ-γ or ε and αδ-δ.

The N-terminal region of AChR α (alpha) represents the main immunogenic region (MIR). The MIR is a cluster of overlapping epitopes rather than one single epitope and epitopes are conformation dependent (Koneczny et al, Cells. 2019 July; 8(7): 671). Approximately half of all MG patients generate autoantibodies against the MIR. The MIR is angled outward from the central axis of the AChR, which prevents the cross-linking of two a subunits within an AChR, and instead induces the cross-linking of adjacent AChRs. MIR-specific antibodies may interfere with the binding of ACh to the ACh binding site, and they may allosterically affect the AChR function. A region of AChR beta also represents an immunogenic region AChR. Other immunogenic regions of the AChR may be found in any one or more or combination of the AChR subunits.

As used herein, “nicotinic acetylcholine receptors”, or nAChRs, are an acetylcholine receptor (AChR) that respond to the neurotransmitter acetylcholine, and also respond to drugs such as the agonist nicotine. They are found in the central and peripheral nervous system, muscle, and many other tissues of many organisms. At the neuromuscular junction they are the primary receptor in muscle for motor nerve-muscle communication that controls muscle contraction. In the peripheral nervous system: (1) they transmit outgoing signals from the presynaptic to the postsynaptic cells within the sympathetic and parasympathetic nervous system, and (2) they are the receptors found on skeletal muscle that receive acetylcholine released to signal for muscular contraction. In the immune system, nAChRs regulate inflammatory processes and signal through distinct intracellular pathways. The nicotinic receptors are considered cholinergic receptors, since they respond to acetylcholine. Nicotinic receptors get their name from nicotine which does not stimulate the muscarinic acetylcholine receptors but selectively binds to the nicotinic receptors instead.

### Chimeric Antigen Receptors and Chimeric Autoantibody Receptors:

As used herein, a “chimeric antigen receptor” (CAR) polypeptide comprises an extracellular antigen-binding domain, comprising an antibody or antibody fragment that binds a target antigen, a transmembrane domain, and an intracellular domain. CARs are typically described as comprising an extracellular ectodomain (antigen-binding domain) derived from an antibody and an endodomain comprising signaling modules derived from T cell signaling proteins. The CAAR of the present invention is based on a CAR structure but employs an autoantigen to direct the CAAR specificity. References to CAR constructs and common knowledge in the context of CAR construct design, for example with respect to the transmembrane and intracellular component, therefore apply to the present invention, if necessary.

In the present invention, the chimeric autoantibody receptors (CAAR) comprise an autoantigen in place of the extracellular antigen-binding domain of a CAR. This autoantigen may be referred to, without limitation, as a targeting domain, binding domain, or an extracellular autoantibody-binding domain, or as an extracellular ectodomain.

In a preferred embodiment, the ectodomain preferably comprises an autoantigen of a nicotinic acetylcholine receptor (nAChR) or fragment thereof.

The autoantigen may be attached to a hinge region that provides flexibility and transduces signals through an anchoring transmembrane moiety to an intracellular signaling domain.

The transmembrane domains originate preferably from either CD8a or CD28. In the first generation of CARs the signaling domain consists of the zeta chain of the TCR complex. The term “generation” refers to the structure of the intracellular signaling domains. Second generation CARs are equipped with a single costimulatory domain originated from CD28 or 4-1 BB. Third generation CARs already include two costimulatory domains, e.g. CD28, 4-1 BB, ICOS or OX40, CD3 zeta. The present invention preferably relates to a second or third generation “CAR” format, although the autoantibody-binding fragments described herein may be employed in any given CAR format.

In various embodiments, genetically engineered receptors that redirect cytotoxicity of immune effector cells toward B cells are provided. These genetically engineered receptors are referred to herein as CAARs. CAARs are molecules that combine autoantigen-autoantibody specificity for a desired target (B-cell that secretes/presents pathogenic autoantibodies) with a T cell receptor-activating intracellular domain to generate a chimeric protein that exhibits a specific cellular immune activity. As used herein, the term “chimeric” describes being composed of parts of different proteins or DNAs from different origins. The main characteristic of the CAARs described herein are their ability to redirect immune effector cell specificity, thereby triggering the proliferation of antigen-specific effector T cells, cytokine production (such as IFN-γ), and production of molecules that can mediate death of the target B cells expressing the target autoantibody.

### Autoantigen Domain:

The present invention is partly based on the discovery that chimeric autoantibody receptors can be used to target autoantibody-producing B cells that cause autoimmune disease. The invention includes compositions comprising at least one chimeric autoantibody receptor (CAAR) specific for an autoantibody, vectors comprising the same, compositions comprising CAAR vectors packaged in viral particles, and recombinant T cells or other effector cells comprising the CAAR. The invention also includes methods of making a genetically modified T cell expressing a CAAR (CAART) wherein the expressed CAAR comprises an autoantigen of a nicotinic acetylcholine receptor (nAChR) or fragment thereof.

The “extracellular antigen-binding domain” or “extracellular binding domain” or “targeting domain” or “autoantigen” are used interchangeably and provide a CAAR with the ability to specifically bind to the target autoantibody of interest. The binding domain may be derived either from a natural, synthetic, semi-synthetic, or recombinant source. Multiple examples of the autoantigen domain are presented herein.

“Specific binding” is to be understood as via one skilled in the art, whereby the skilled person is clearly aware of various experimental procedures that can be used to test binding and binding specificity. Methods for determining equilibrium association or equilibrium dissociation constants are known in the art. Some cross-reaction or background binding may be inevitable in many protein-protein interactions; this is not to detract from the “specificity” of the binding between CAAR and autoantibody. “Specific binding” describes binding of an autoantigen to an autoantibody at greater binding affinity than background (unspecific) binding. The term “directed against” is also applicable when considering the term “specificity” in understanding the interaction between antibody and epitope.

An “antigen (Ag)” refers to a compound, composition, or substance that can stimulate the production of antibodies or a T cell response in an animal. An “epitope” refers to the region of an antigen to which an antibody binds. Epitopes can be formed both from contiguous amino acids or noncontiguous amino acids juxtaposed by tertiary folding of a protein.

By “autoantigen” is meant an endogenous antigen that stimulates production of an autoimmune response, such as production of autoantibodies. Autoantigen also includes a self-antigen or antigen from a normal tissue that is the target of a cell-mediated or an antibody-mediated immune response that may result in the development of an autoimmune disease. “Autoantibody” refers to an antibody that is produced by a B cell specific for an autoantigen.

An illustrative example of the autoantigen component of the CAARs contemplated herein include but are not limited to the sequences set forth in SEQ ID NOs 1 to 12.

### Antibodies and Antibody Fragments:

The CAAR of the present invention preferably does not comprise an extracellular antigen-binding domain comprising an antibody or antibody fragment. The present CAAR construct is therefore distinct from common CAR constructs.

As used herein, an “antibody” generally refers to a protein consisting of one or more polypeptides substantially encoded by immunoglobulin genes or fragments of immunoglobulin genes. Where the term “antibody” is used, the term “antibody fragment” may also be considered to be referred to. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. The basic immunoglobulin (antibody) structural unit is known to comprise a tetramer or dimer. Each tetramer is composed of two identical pairs of polypeptide chains, each pair having one “light” (L) (about 25 kD) and one “heavy” (H) chain (about 50-70 kD). The N-terminus of each chain defines a variable region of about 100 to 110 or more amino acids, primarily responsible for antigen recognition. The terms “variable light chain” and “variable heavy chain” refer to these variable regions of the light and heavy chains respectively.

The CAARs of the invention are intended to bind against mammalian, in particular human, autoantibody targets. The use of protein names, for example defining the autoantigen of the CAAR construct, may correspond to either mouse or human versions of a protein.

### Additional Components of the CAAR

In certain embodiments, the CAARs contemplated herein may comprise linker residues between the various domains, added for appropriate spacing and conformation of the molecule, for example a linker comprising an amino acid sequence that connects the extracellular and transmembrane domains, or fragments of an autoantigen. CAARs contemplated herein, may comprise one, two, three, four, or five or more linkers. In particular embodiments, the length of a linker is about 1 to about 25 amino acids, about 5 to about 20 amino acids, or about 10 to about 20 amino acids, or any intervening length of amino acids.

Illustrative examples of linkers include glycine polymers; glycine-serine polymers; glycine-alanine polymers; alanine-serine polymers; and other flexible linkers known in the art, such as the Whitlow linker. Glycine and glycine-serine polymers are relatively unstructured, and therefore may be able to serve as a neutral tether between domains of fusion proteins such as the CAARs described herein.

In particular embodiments, the binding domain of the CAAR is followed by one or more “linkers”, “spacers” or “linker polypeptides” or “spacer polypeptides”, which refers in some embodiments to a region that moves the autoantibody binding domain away from the effector cell surface to enable proper contact, antigen binding and immune cell activation. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region. In one embodiment, the spacer domain comprises the CH2 and CH3 domains of IgG1 or IgG4.

The extracellular binding domain of the CAAR may in some embodiments be followed by one or more “hinge domains,” which play a role in positioning the binding domain away from the effector cell surface to enable proper cell/cell contact, antigen binding and activation. A CAAR may comprise one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region. Illustrative hinge domains suitable for use in the CAARs described herein include the hinge region derived from the extracellular regions of type 1 membrane proteins such as CD8 alpha, CD4, CD28, PD1, CD 152, and CD7, which may be wild-type hinge regions from these molecules or may be altered. In another embodiment, the hinge domain comprises a PD1, CD 152, or CD8 alpha hinge region.

The “transmembrane domain” is the portion of the CAAR that fuses the extracellular binding portion and intracellular signaling domain and anchors the CAAR to the plasma membrane of the immune effector cell.

The TM domain may be derived either from a natural, synthetic, semi-synthetic, or recombinant source. The TM domain may be derived from the alpha, beta or zeta chain of the T-cell receptor, CD3c, CD3, CD4, CD5, CD8 alpha, CD9, CD 16, CD22, CD27, CD28, CD33, CD37, CD45, CD64, CD80, CD86, CD 134, CD 137, CD 152, CD 154, and PD1. In one embodiment, the CAARs contemplated herein comprise a TM domain derived from CD8 alpha or CD28.

In particular embodiments, CAARs contemplated herein comprise an intracellular signaling domain. An “intracellular signaling domain,” refers to the part of a CAAR that participates in transducing the message of effective CAAR binding to a target autoantibody into the interior of the immune effector cell to elicit effector cell function, e.g., activation, cytokine production, proliferation and cytotoxic activity, including the release of cytotoxic factors to the CAAR-bound target, or other cellular responses elicited with antigen binding to the extracellular CAAR domain.

The term “effector function” refers to a specialized function of an immune effector cell. Effector function of the T cell, for example, may be cytolytic activity or help or activity including the secretion of a cytokine. Thus, the term “intracellular signaling domain” refers to the portion of a protein which transduces the effector function signal and that directs the cell to perform a specialized function.

CAARs contemplated herein comprise one or more co-stimulatory signaling domains to enhance the efficacy, expansion and/or memory formation of T cells expressing CAAR receptors. As used herein, the term, “co-stimulatory signaling domain” refers to an intracellular signaling domain of a co-stimulatory molecule. Co-stimulatory molecules are cell surface molecules other than antigen receptors or Fc receptors that provide a second signal required for efficient activation and function of T lymphocytes upon binding to the target.

### Polypeptides

“Peptide”, “polypeptide”, “polypeptide fragment” and “protein” are used interchangeably, unless specified to the contrary, and according to conventional meaning, i.e., as a sequence of amino acids. Polypeptides are not limited to a specific length, e.g., they may comprise a full length protein sequence or a fragment of a full length protein, and may include post-translational modifications of the polypeptide, for example, glycosylations, acetylations, phosphorylations and the like, as well as other modifications known in the art, both naturally occurring and non-naturally occurring.

In various embodiments, the CAAR polypeptides contemplated herein comprise a signal (or leader) sequence at the N-terminal end of the protein, which co-translationally or post-translationally directs transfer of the protein. Polypeptides can be prepared using any of a variety of well-known recombinant and/or synthetic techniques. Polypeptides contemplated herein specifically encompass the CAARs of the present disclosure, or sequences that have deletions from, additions to, and/or substitutions of one or more amino acid of a CAAR as disclosed herein.

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances. Similarly, an “isolated cell” refers to a cell that has been obtained from an in vivo tissue or organ and is substantially free of extracellular matrix.

### Nucleic Acids

As used herein, the terms “polynucleotide” or “nucleic acid molecule” refers to any nucleic acid molecule, for example DNA or RNA, such as messenger RNA (mRNA), RNA, genomic RNA (gRNA), plus strand RNA (RNA(+)), minus strand RNA (RNA(−)), genomic DNA (gDNA), complementary DNA (cDNA) or recombinant DNA. Polynucleotides include single and double stranded polynucleotides. Preferably, polynucleotides of the invention include polynucleotides or variants having at least about 50%, 55%, 60%, 65%, 70%, 75%, 80%, 85%, 90%, 95%, 96%, 97%, 98%, 99% or 100% sequence identity to any of the reference sequences described herein, typically where the variant maintains at least one biological activity of the reference sequence. In various illustrative embodiments, the present invention contemplates, in part, polynucleotides comprising expression vectors, viral vectors, and transfer plasmids, and compositions, and cells comprising the same.

Polynucleotides can be prepared, manipulated and/or expressed using any of a variety of well-established techniques known and available in the art. In order to express a desired polypeptide, a nucleotide sequence encoding the polypeptide, can be inserted into appropriate vector. Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or MI 3 phage, and animal viruses. Examples of categories of animal viruses useful as vectors include, without limitation, retrovirus (including lentivirus), adenovirus, adeno-associated virus, herpesvirus {e.g., herpes simplex virus), poxvirus, baculovirus, papillomavirus, and papovavirus (e.g., SV40). Examples of expression vectors are pCIneo vectors (Promega) for expression in mammalian cells; pLenti4/V5-DEST™, pLenti6/V5-DEST™, and pLenti6.2N5-GW/lacZ (Invitrogen) for lentivirus-mediated gene transfer and expression in mammalian cells. In particular embodiments, the coding sequences of the chimeric proteins disclosed herein can be ligated into such expression vectors for the expression of the chimeric protein in mammalian cells. The “control elements” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

### Vectors

In particular embodiments, a cell (e.g., an immune effector cell, such as a T cell) is transduced with a retroviral vector, e.g., gamma-retroviral or a lentiviral vector, encoding a CAAR.

Retroviruses are a common tool for gene delivery. In particular embodiments, a retrovirus is used to deliver a polynucleotide encoding a CAAR to a cell. As used herein, the term “retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are envisaged. In particular embodiments, a lentivirus is used to deliver a polynucleotide comprising a CAAR to a cell.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus.

In a preferred embodiment the invention therefore relates to a method for transfecting cells with an expression vector encoding a CAAR. For example, in some embodiments, the vector comprises additional sequences, such as sequences that facilitate expression of the CAAR, such a promoter, enhancer, poly-A signal or Woodchuck Hepatitis Virus (WHP) Posttranscriptional Regulatory Element (WPRE), and/or one or more introns. In preferred embodiments, the CAAR-coding sequence is flanked by transposon sequences, such that the presence of a transposase allows the coding sequence to integrate into the genome of the transfected cell.

In some embodiments, the genetically transformed cells are further transfected with a transposase that facilitates integration of a CAAR coding sequence into the genome of the transfected cells. In some embodiments the transposase is provided as DNA expression vector. However, in preferred embodiments, the transposase is provided as an expressible RNA or a protein such that long-term expression of the transposase does not occur in the transgenic cells. For example, in some embodiments, the transposase is provided as an mRNA (e.g., an mRNA comprising a cap and poly-A tail). Any transposase system may be used in accordance with the embodiments of the present invention. However, in some embodiments, the transposase is salmonid-type Tel-like transposase (SB). For example, the transposase can be the so called “Sleeping beauty” transposase, see e.g., U.S. Pat. No. 6,489,458, incorporated herein by reference. In some embodiments, the transposase is an engineered enzyme with increased enzymatic activity. Some specific examples of transposases include, without limitation, SB 10, SB 11 or SB 100× transposase (see, e.g., Mates et al, 2009, Nat Genet. 41(6):753-61, or U.S. Pat. No. 9,228,180, herein incorporated by reference). For example, a method can involve electroporation of cells with an mRNA encoding an SB 10, SB 11 or SB 100× transposase.

Transposable elements are natural, non-viral gene delivery vehicles capable of mediating stable genomic integration. The Sleeping Beauty (SB) transposon has the ability to cut-and-paste a nucleic acid sequence of interest into the genome, providing the basis for long-term, permanent transgene expression in transgenic cells and organisms, in this case for the transformation of immune cells, preferably T cells, with the CAAR-encoding nucleic acid sequences of the present invention. The SB transposon system is relatively well characterized and has been extensively engineered for efficient gene delivery and gene discovery purposes in a wide range of vertebrates, including humans. A skilled person is capable of identifying appropriate variants of the SB systema and incorporating these into the invention as is necessary. Specific, non-limiting, examples are provided below. The SB system is a safe and simple-to-use vector that enables cost-effective, rapid preparation of therapeutic doses of cell products.

Generally, a transposon system includes a transposon and a transposase. The transposon acts as a carrier, which carries the gene to be inserted into the genome. The transposase is the so-called “workhorse” of the system, catalyzing the process of transposition. The transposase is located between the inverted terminal repeats (ITRs) of the transposon. Importantly, the transposase gene can be replaced with any nucleic acid sequence of interest, and the transposase can govern transposition events when encoded by a separate plasmid in trans. Physical separation of the transposon from the transposase enabled optimization of transposon versus transposase ratio, and also provided the freedom of supplying the transposase in the form of mRNA, instead of DNA. First, the transposase recognizes the transposon, and binds the ITRs. During synaptic complex formation, the transposon ends are brought together by transposase monomers (presumably forming a tetramer). The transposase generates a DNA double-strand break upon excision, while single-stranded gaps at the integration site. The pre-integration complex containing the transposon bound transposase performs the integration into the host genome. SB transposition is a highly coordinated reaction that efficiently filters out abnormal, toxic transposition intermediates (reviewed in Narayanavari & Izsvák, Cell & Gene Therapy insights, 2017).

Previous optimization of nucleotide residues (including mutations, deletions and additions) within the ITRs of the original SB transposon (pT) resulted in improved transposon versions, such as pT2, pT3, pT2B and pT4, which may be employed for the CAAR-encoding sequences described herein. In one embodiment, pT4 is employed.

Previous screening involving mutagenizing the primary amino acid sequence of the SB transposase has provided a number of hyperactive transposase versions. SB100× is 100-fold hyperactive compared to the originally resurrected transposase (SB10) in certain cell types. Currently available SB transposases include, but are not limited to, SB10, SB11 (3-fold higher activity than SB10), SB12 (4-fold higher than SB10), HSB1-HSB5 (up to 10-fold higher than SB10), HSB13-HSB17 (HSB17 is 17-fold higher than SB10), SB100× (100-fold higher than SB10), SB150× (130-fold higher than SB10). In one embodiment, SB100× is employed.

A further aspect of the invention relates to a genetically modified immune cell comprising a nucleic acid molecule or vector as described herein, and/or expressing a CAAR as described herein.

A further aspect of the invention relates to a vector comprising a nucleic acid molecule as described herein, preferably a viral vector, more preferably a gamma retroviral vector. In another aspect of the invention, the invention relates to a transposon vector, preferably a sleeping beauty vector, encoding and preferably capable of expressing the inventive CAAR.

In a preferred embodiment the immune cells intended for administering in treatment of the diseases mentioned herein are genetically modified with a nucleic acid as described herein, encoding and expressing the CAAR as described herein, using a “Sleeping beauty” transposon system, in particular a sleeping beauty transposase. The Sleeping Beauty transposon system is a synthetic DNA transposon designed to introduce precisely defined DNA sequences into the chromosomes of vertebrate animals, in the context of the present invention for the purposes of modifying immune cells to express the CAAR as described herein. The sleeping beauty transposons combine the advantages of viruses and naked DNA. Viruses have been evolutionarily selected based on their abilities to infect and replicate in new host cells. Simultaneously, cells have evolved major molecular defense mechanisms to protect themselves against viral infections. Avoiding the use of viruses is also important for social and regulatory reasons. The use of non-viral vectors such as the sleeping beauty system therefore avoids many, but not all, of the defenses that cells employ against vectors. For this reason, the sleeping beauty system enables particularly effective and safe genetic modification of the immune cells for administration to a patient.

### Sequence Variants:

Sequence variants of the claimed nucleic acids, proteins, antibodies, antibody fragments and/or CAARs, for example those defined by % sequence identity, that maintain similar binding properties of the invention are also included in the scope of the invention. Such variants, which show alternative sequences, but maintain essentially the same binding properties, such as target specificity, as the specific sequences provided are known as functional analogues, or as functionally analogous. Sequence identity relates to the percentage of identical nucleotides or amino acids when carrying out a sequence alignment.

The recitation “sequence identity” as used herein refers to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

It will be appreciated by those of ordinary skill in the art that, as a result of the degeneracy of the genetic code, there are many nucleotide sequences that encode a polypeptide as described herein. Some of these polynucleotides bear minimal homology or sequence identity to the nucleotide sequence of any native gene. Nonetheless, polynucleotides that vary due to differences in codon usage are specifically contemplated by the present invention. Deletions, substitutions and other changes in sequence that fall under the described sequence identity are also encompassed in the invention.

Protein sequence modifications, which may occur through substitutions, are also included within the scope of the invention. Substitutions as defined herein are modifications made to the amino acid sequence of the protein, whereby one or more amino acids are replaced with the same number of (different) amino acids, producing a protein which contains a different amino acid sequence than the primary protein. Substitutions may be carried out that preferably do not significantly alter the function of the protein. Like additions, substitutions may be natural or artificial. It is well known in the art that amino acid substitutions may be made without significantly altering the protein's function. This is particularly true when the modification relates to a “conservative” amino acid substitution, which is the substitution of one amino acid for another of similar properties. Such “conserved” amino acids can be natural or synthetic amino acids which because of size, charge, polarity and conformation can be substituted without significantly affecting the structure and function of the protein. Frequently, many amino acids may be substituted by conservative amino acids without deleteriously affecting the protein's function.

In general, the non-polar amino acids Gly, Ala, Val, Ile and Leu; the non-polar aromatic amino acids Phe, Trp and Tyr; the neutral polar amino acids Ser, Thr, Cys, Gln, Asn and Met; the positively charged amino acids Lys, Arg and His; the negatively charged amino acids Asp and Glu, represent groups of conservative amino acids. This list is not exhaustive. For example, it is well known that Ala, Gly, Ser and sometimes Cys can substitute for each other even though they belong to different groups.

Substitution variants have at least one amino acid residue in the antibody molecule removed and a different residue inserted in its place. The sites of greatest interest for substitutional mutagenesis include the hypervariable regions, but FR alterations are also contemplated. If such substitutions result in a change in biological activity, then more substantial changes, denominated “exemplary substitutions” in the table immediately below, or as further described below in reference to amino acid classes, may be introduced and the products screened.

### Potential Amino Acid Substitutions:

| | Preferred | |
|---|---|---|
| Original | conservative | |
| residue | substitutions | Examples of exemplary substitutions |
| Ala (A) | Val | Val; Leu; Ile |
| Asg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn, Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; Norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

Conservative amino acid substitutions are not limited to naturally occurring amino acids, but also include synthetic amino acids. Commonly used synthetic amino acids are omega amino acids of various chain lengths and cyclohexyl alanine which are neutral non-polar analogs; citrulline and methionine sulfoxide which are neutral non-polar analogs, phenylglycine which is an aromatic neutral analog; cysteic acid which is a negatively charged analog and ornithine which is a positively charged amino acid analog. Like the naturally occurring amino acids, this list is not exhaustive, but merely exemplary of the substitutions that are well known in the art.

### Genetically Modified Cells and Immune Cells

The present invention contemplates, in particular embodiments, cells genetically modified to express the CAARs contemplated herein, for use in the treatment of B cell related conditions. As used herein, the term “genetically engineered” or “genetically modified” refers to the addition of extra genetic material in the form of DNA or RNA into the total genetic material in a cell. The terms, “genetically modified cells,” “modified cells,” and, “redirected cells,” are used interchangeably.

An “immune cell” or “immune effector cell” is any cell of the immune system that has one or more effector functions (e.g., cytotoxic cell killing activity, secretion of cytokines, induction of ADCC and/or CDC).

Immune effector cells of the invention can be autologous/autogeneic (“self) or non-autologous (“non-self,” e.g., allogeneic, syngeneic or xenogeneic). “Autologous”, as used herein, refers to cells from the same subject, and represent a preferred embodiment of the invention. “Allogeneic”, as used herein, refers to cells of the same species that differ genetically to the cell in comparison. “Syngeneic”, as used herein, refers to cells of a different subject that are genetically identical to the cell in comparison. “Xenogeneic”, as used herein, refers to cells of a different species to the cell in comparison. In preferred embodiments, the cells of the invention are autologous or allogeneic.

Illustrative immune effector cells used with the CAARs contemplated herein include T lymphocytes. The terms “T cell” or “T lymphocyte” are art-recognized and are intended to include thymocytes, immature T lymphocytes, mature T lymphocytes, resting T lymphocytes, cytokine-induced killer cells (CIK cells) or activated T lymphocytes. Cytokine-induced killer (CIK) cells are typically CD3- and CD56-positive, non-major histocompatibility complex (MHC)-restricted, natural killer (NK)-like T lymphocytes. A T cell can be a T helper (Th; CD4+ T cell) cell, for example a T helper 1 (Th1) or a T helper 2 (Th2) cell. The T cell can be a cytotoxic T cell (CTL; CD8⁺ T cell), CD4⁺CD8⁺ T cell, CD4 CD8 T cell, or any other subset of T cells. Other illustrative populations of T cells suitable for use in particular embodiments include naive T cells and memory T cells.

For example, when reintroduced back to patients after autologous cell transplantation, the T cells modified with the CAAR of the invention as described herein may recognize and kill pathogenic autoantibody-producing B cells. CIK cells may have enhanced cytotoxic activity compared to other T cells, and therefore represent a preferred embodiment of an immune cell of the present invention.

As would be understood by the skilled person, other cells may also be used as immune effector cells with the CAARs as described herein. In particular, immune effector cells also include NK cells, NKT cells, neutrophils, and macrophages. Immune effector cells also include progenitors of effector cells wherein such progenitor cells can be induced to differentiate into an immune effector cells in vivo or in vitro.

The present invention provides methods for making the immune effector cells which express the CAAR contemplated herein. In one embodiment, the method comprises transfecting or transducing immune effector cells isolated from an individual such that the immune effector cells express one or more CAAR as described herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a CAAR. In this regard, the immune effector cells may be cultured before and/or after being genetically modified (i.e., transduced or transfected to express a CAAR contemplated herein).

In particular embodiments, prior to in vitro manipulation or genetic modification of the immune effector cells described herein, the source of cells is obtained from a subject. In particular embodiments, the CAAR-modified immune effector cells comprise T cells. T cells can be obtained from a number of sources including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation, antibody-conjugated bead-based methods such as MACS™ separation (Miltenyi). In one embodiment, cells from the circulating blood of an individual are obtained by apheresis. The apheresis product typically contains lymphocytes, including T cells, monocytes, granulocyte, B cells, other nucleated white blood cells, red blood cells, and platelets. In one embodiment, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. As would be appreciated by those of ordinary skill in the art, a washing step may be accomplished by methods known to those in the art, such as by using a semiautomated flow through centrifuge. For example, the Cobe 2991 cell processor, the Baxter CytoMate, or the like. After washing, the cells may be resuspended in a variety of biocompatible buffers or other saline solution with or without buffer. In certain embodiments, the undesirable components of the apheresis sample may be removed in the cell directly resuspended culture media.

In certain embodiments, T cells are isolated from peripheral blood mononuclear cells (PBMCs) by lysing the red blood cells and depleting the monocytes, for example, by centrifugation through a PERCOLL™ gradient. A specific subpopulation of T cells can be further isolated by positive or negative selection techniques. One method for use herein is cell sorting and/or selection via negative magnetic immunoadherence or flow cytometry that uses a cocktail of monoclonal antibodies directed to cell surface markers present on the cells negatively selected.

PBMC may be directly genetically modified to express CAARs using methods contemplated herein. In certain embodiments, after isolation of PBMC, T lymphocytes are further isolated and in certain embodiments, both cytotoxic and helper T lymphocytes can be sorted into naive, memory, and effector T cell subpopulations either before or after genetic modification and/or expansion. CD8⁺ cells can be obtained by using standard methods. In some embodiments, CD8⁺ cells are further sorted into naive, central memory, and effector cells by identifying cell surface antigens that are associated with each of those types of CD8⁺ cells.

The immune effector cells, such as T cells, can be genetically modified following isolation using known methods, or the immune effector cells can be activated and expanded (or differentiated in the case of progenitors) in vitro prior to being genetically modified. In a particular embodiment, the immune effector cells, such as T cells, are genetically modified with the chimeric antigen receptors contemplated herein (e.g., transduced with a viral vector comprising a nucleic acid encoding a CAAR) and then are activated and expanded in vitro. In various embodiments, T cells can be activated and expanded before or after genetic modification to express a CAAR, using methods as described, for example, in U.S. Pat. Nos. 6,352,694; 6,534,055; 6,905,680; 6,692,964; 5,858,358; 6,887,466; 6,905,681; 7, 144,575; 7,067,318; 7, 172,869; 7,232,566; 7, 175,843; 5,883,223; 6,905,874; 6,797,514; 6,867,041; and U.S. Patent Application Publication No. 20060121005.

In a further embodiment, a mixture of, e.g., one, two, three, four, five or more, different expression vectors can be used in genetically modifying a donor population of immune effector cells wherein each vector encodes a different chimeric antigen receptor protein as contemplated herein. The resulting modified immune effector cells forms a mixed population of modified cells, with a proportion of the modified cells expressing more than one different CAAR proteins.

In one embodiment, the invention provides a method of storing genetically modified murine, human or humanized CAAR protein expressing immune effector cells which target an autoantibody, comprising cryopreserving the immune effector cells such that the cells remain viable upon thawing. A fraction of the immune effector cells expressing the CAAR proteins can be cryopreserved by methods known in the art to provide a permanent source of such cells for the future treatment of patients afflicted with the B cell related condition. When needed, the cryopreserved transformed immune effector cells can be thawed, grown and expanded for more such cells.

In one embodiment the immune cell is preferably selected from the group consisting of a T lymphocyte or an NK cell, more preferably cytotoxic T lymphocytes.

In a preferred embodiment the genetically modified immune cell comprising a nucleic acid molecule or vector as described herein, and/or expressing a CAAR as described herein, is characterised in that it is CD4⁺ and/or CD8⁺ T cell, preferably a mixture of CD4+ and CD8+ T cells. These T cell populations, and preferably the composition comprising both CD4⁺ and CD8⁺ transformed cells, show particularly effective cytolytic activity against various B cells, preferably against those cells and/or the associated medical conditions described herein.

In a preferred embodiment the genetically modified immune cells comprising a nucleic acid molecule or vector as described herein, and/or expressing a CAAR as described herein, are CD4⁺ and CD8⁺ T cells, preferably in a ration of 1:10 to 10:1, more preferably in a ratio of 5:1 to 1:5, 2:1 to 1:2 or 1:1. Administration of modified CAAR-T cells expressing the CAAR described herein at the ratios mentioned, preferably at a 1:1 CD4⁺/CD8⁺ ratio, lead to beneficial characteristics during treatment of the diseases mentioned herein, for example these ratios lead to improved therapeutic response and reduced toxicity.

### Compositions and Formulations

The compositions contemplated herein may comprise one or more polypeptides, polynucleotides, vectors comprising said polynucleotides, genetically modified immune effector cells, etc., as described and contemplated herein. Compositions include but are not limited to pharmaceutical compositions.

A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions of the invention may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular embodiments, compositions of the present invention comprise an amount of CAAR-expressing immune effector cells contemplated herein. As used herein, the term “amount” refers to “an amount effective” or “an effective amount” of a genetically modified therapeutic cell, e.g., T cell, to achieve a beneficial or desired prophylactic or therapeutic result, including clinical results.

A “prophylactically effective amount” refers to an amount of a genetically modified therapeutic cell effective to achieve the desired prophylactic result. Typically, but not necessarily, since a prophylactic dose is used in subjects prior to or at an earlier stage of disease, the prophylactically effective amount is less than the therapeutically effective amount. The term prophylactic does not necessarily refer to a complete prohibition or prevention of a particular medical disorder. The term prophylactic also refers to the reduction of risk of a certain medical disorder occurring or worsening in its symptoms.

A “therapeutically effective amount” of a genetically modified therapeutic cell may vary according to factors such as the disease state, age, sex, and weight of the individual, and the ability of the stem and progenitor cells to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject (e.g., a patient). When a therapeutic amount is indicated, the precise amount of the compositions of the present invention to be administered can be determined by a physician with consideration of individual differences in age, weight, tumor size, extent of infection or metastasis, and condition of the patient (subject).

It can generally be stated that a pharmaceutical composition comprising the immune cells (T cells) described herein may be administered at a dosage of 10² to 10¹⁰ cells/kg body weight, preferably 10⁵ to 10⁶ cells/kg body weight, including all integer values within those ranges. The number of cells will depend upon the ultimate use for which the composition is intended as will the type of cells included therein. For uses provided herein, the cells are generally in a volume of a liter or less, can be 500 mLs or less, even 250 mLs or 100 mLs or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present invention, particularly since all the infused cells will be redirected to a particular target antigen, lower numbers of cells may be administered. CAAR expressing cell compositions may be administered multiple times at dosages within these ranges. The cells may be allogeneic, syngeneic, xenogeneic, or autologous to the patient undergoing therapy.

Generally, compositions comprising the cells activated and expanded as described herein may be utilized in the treatment and prevention of diseases that arise in individuals who are immunocompromised. The CAAR-modified T cells of the present invention may be administered either alone, or as a pharmaceutical composition in combination with carriers, diluents, excipients, and/or with other components such as IL-2 or other cytokines or cell populations. In particular embodiments, pharmaceutical compositions contemplated herein comprise an amount of genetically modified T cells, in combination with one or more pharmaceutically or physiologically acceptable carriers, diluents or excipients.

Pharmaceutical compositions of the present invention comprising a CAAR-expressing immune effector cell population, such as T cells, may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for parenteral administration, e.g., intravascular (intravenous or intraarterial), intraperitoneal or intramuscular administration.

The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In a particular embodiment, compositions contemplated herein comprise an effective amount of CAAR-expressing immune effector cells, alone or in combination with one or more therapeutic agents. Thus, the CAAR-expressing immune effector cell compositions may be administered alone or in combination with other known treatments, such as other immunotherapies, etc. The compositions may also be administered in combination with antibiotics. Such therapeutic agents may be accepted in the art as a standard treatment for a particular disease state as described herein, such as a particular cancer. Exemplary therapeutic agents contemplated include cytokines, growth factors, steroids, NSAIDs, DMARDs, anti-inflammatories, chemotherapeutics, radiotherapeutics, therapeutic antibodies, or other active and ancillary agents.

### Therapeutic Methods

As used herein, the terms “individual” and “subject” are often used interchangeably and refer to any animal that exhibits a symptom of a disease, disorder, or condition that can be treated with the gene therapy vectors, cell-based therapeutics, and methods disclosed elsewhere herein. In preferred embodiments, a subject includes any animal that exhibits symptoms of a disease, disorder, or condition of the hematopoietic system, e.g., an autoimmune disease, that can be treated with the cell-based therapeutics and methods disclosed herein. Suitable subjects include laboratory animals (such as mouse, rat, rabbit, or guinea pig), farm animals, and domestic animals or pets (such as a cat or dog). Non-human primates and, preferably, human patients, are included.

As used herein “treatment” or “treating” includes any beneficial or desirable effect on the symptoms or pathology of a disease or pathological condition, and may include even minimal reductions in one or more measurable markers of the disease or condition being treated. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent” and similar words such as “prevented”, “preventing” or “prophylactic” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

The quantity and frequency of administration will be determined by such factors as the condition of the patient, and the type and severity of the patient's disease, although appropriate dosages may be determined by clinical trials.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

### FIGURES

The invention is demonstrated by way of example by the following figures. The figures are to be considered as providing a further description of potentially preferred embodiments that enhance the support of one or more non-limiting embodiments of the invention.

### SHORT DESCRIPTION OF THE FIGURES

FIG. 1: Schematic representation of the CAAR-T principle.

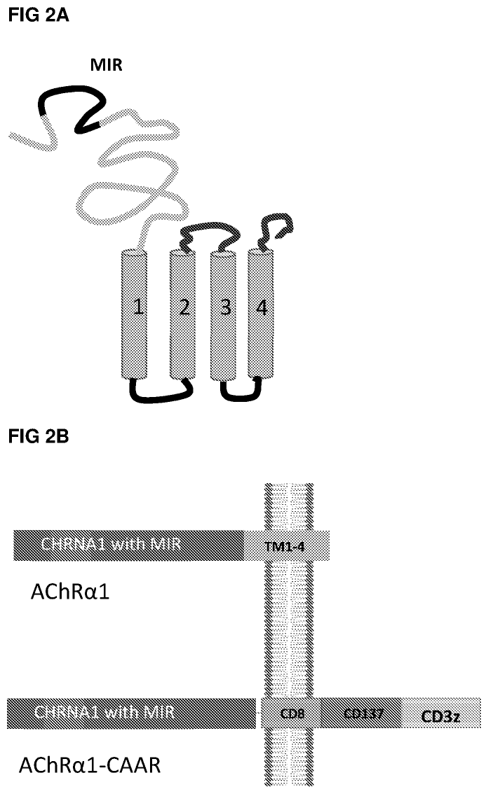

FIG. 2: Diagram of the alpha-1 subunit of nicotinic AChR and CAAR.

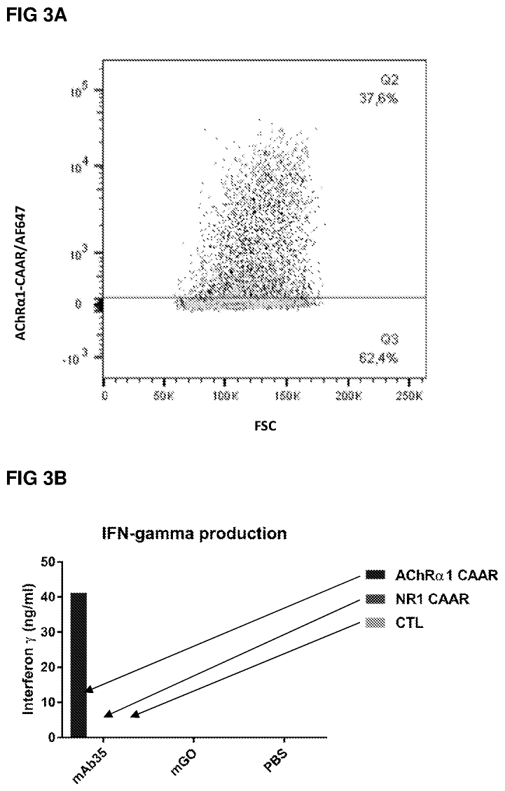

FIG. 3: Expression and functionality of the AChRa1-CAAR construct in human T cells.

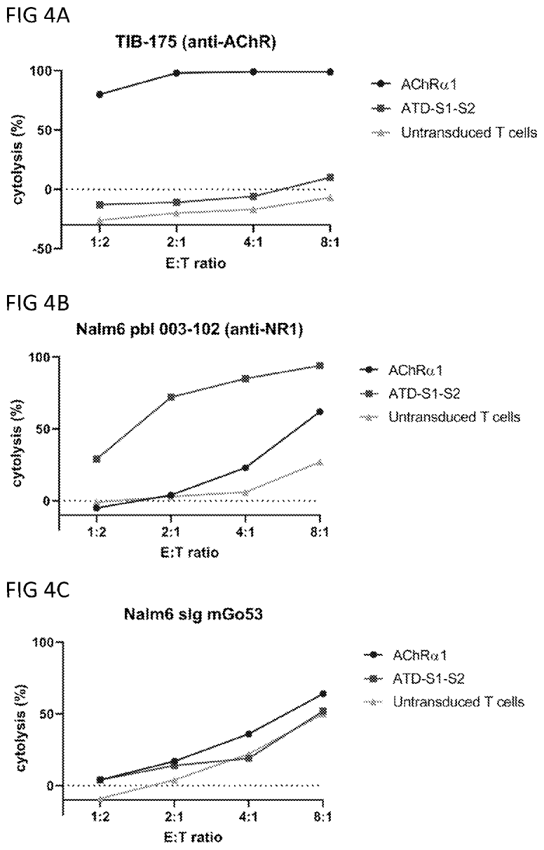

FIG. 4: Specific cytolysis of anti-AChR producing target cells by AChRa1-CAAR T cells.

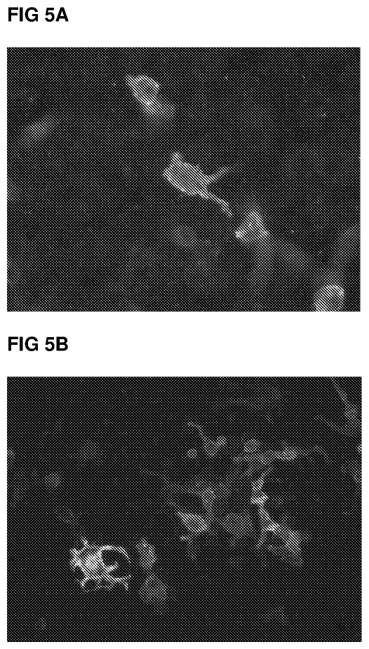

FIG. 5: Expression of CAAR constructs in HEK293-Zellen.

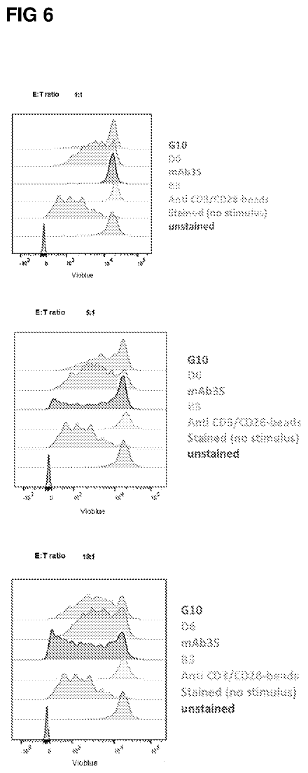

FIG. 6: Proliferation of AChRa1-CAAR T cells.

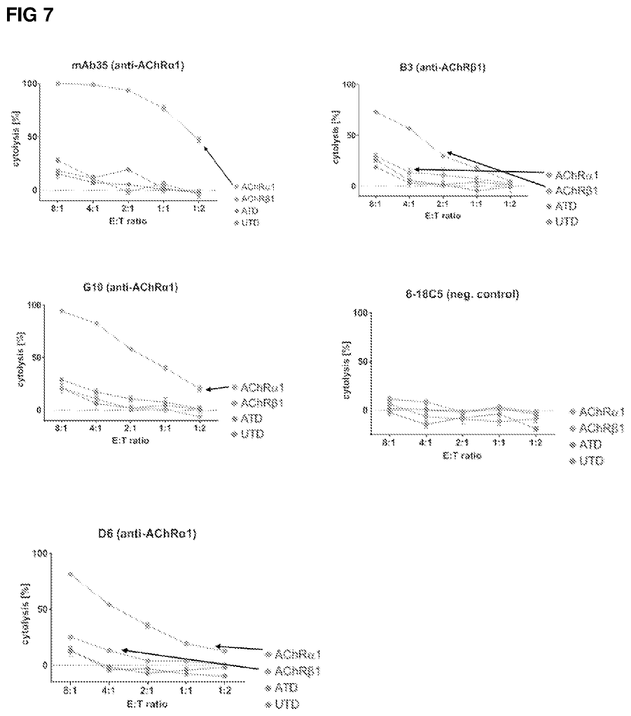

FIG. 7: Cytolytic activity of AChRa1- and AChRb1-CAAR T cells.

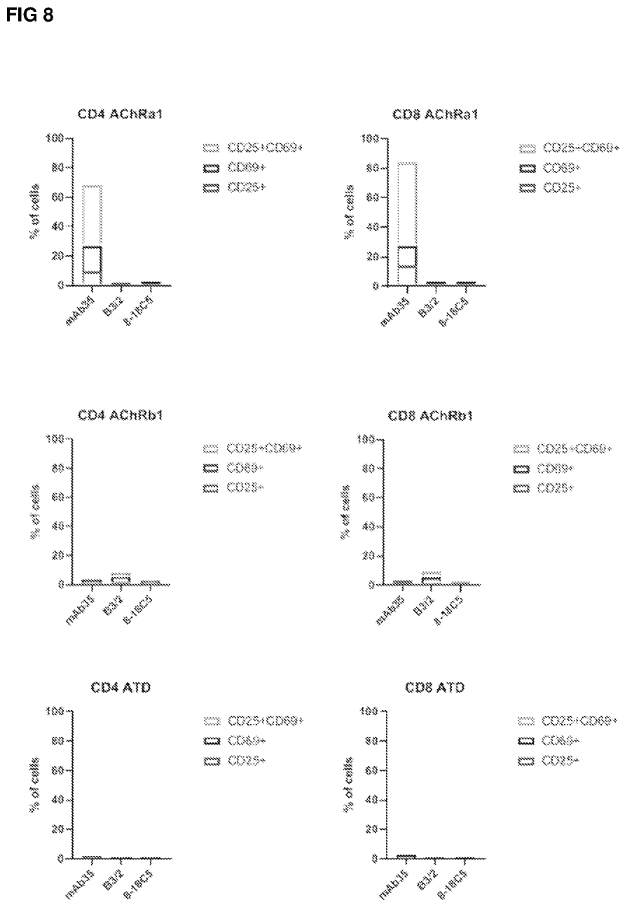

FIG. 8: Expression of activation markers by CAAR T cells after stimulation with antibodies.

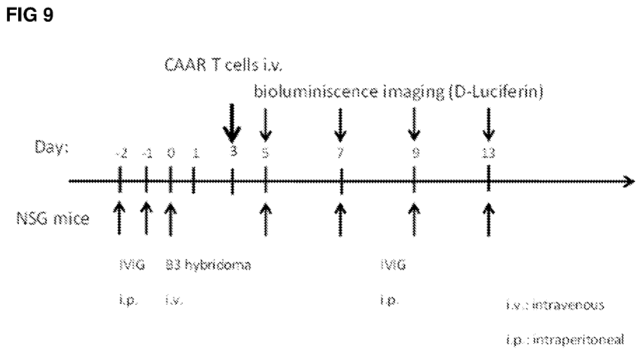

FIG. 9: Experimental plan for in vivo assessment of AChRb1-CAAR T cells.

### DETAILED DESCRIPTION OF THE FIGURES

FIG. 1: Schematic representation of the CAAR-T principle using AChR autoantibody-producing B cells as an example.

FIG. 2: Diagram of the alpha-1 subunit of nicotinic AChR and CAAR. (A) Schematic diagram of the alpha-1 subunit of nicotinic AChR (nAChR). (B) Comparison of the alpha-1 subunit of nAChR with AChRa1-CAAR. MIR=main immunogenic region (black), TM=transmembrane domain.

FIG. 3: Expression and functionality of the AChR-CAAR construct AchRa1-CAAR in human T cells. (A) Flow cytometric analysis of primary human T cells transduced with AChRa1-CAAR shows a surface expression of 37.6% (stained with commercial antibody mAb35 directed against AChR). (B) The wells of a 96-well plate were coated with a monoclonal pathogenic AChR antibody (mAb35) and a control antibody (mGO). 50.000 CAAR T cells were incubated for 48 hours. Activated AChRa1-CAAR T cells released highly specific large amounts of interferon-y (blue). None of the control conditions showed interferon-y release (red, green, values below the detection limit).

FIG. 4: Specific cytolysis of anti-AChR producing target cells by AChRa1-CAAR T cells. AChRa1-CAAR T cells (AChRa1) incubated for 19 h in different effector-target ratios (E:T ratio) with anti-AChR model B cell lines. ATD-S1-S2-CAAR T cells (ATD-S1-S2), whose cytolytic ability could already be demonstrated in a different project unrelated to nAChR, served as positive control.

FIG. 5: Expression of CAAR constructs in HEK293-Zellen. HEK293 cells were transiently transfected with the plasmid DNA of the CAAR constructs (A: AChRγ-CAAR, B: AChRα1β1-CAAR) and the expression was detected by staining with mAb131 (γ-specific) and mAb35 (α-specific).

FIG. 6: Proliferation of AChRa1-CAAR T cells. Co-culture of AChRa1-CAAR T cells was carried out together with alpha- and with beta-specific hybridoma cells. Beforehand, CAAR T cells were stained with CellTrace™ Violet Cell Proliferation Kit. Strong proliferation of CAAR T cells was observed when incubated with alpha-specific hybridoma cells, but not with beta-specific hybridomas.

FIG. 7: Cytolytic activity of AChRa1- and AChRb1-CAAR T cells. AChRa1- and AChRb1-CAAR T cells deplete the respective target cells (hybridomas) within 18 h in a dose-dependent manner. Control hybridomas (8-18C5) are not targeted by CAAR T cells.

FIG. 8: Expression of activation markers by CAAR T cells. After co-culture in a E:T ratio of 1:1 for hours, (CD4+ and CD8+) CAAR T cells express activation markers CD25 and CD69 after co-culture with respective hybridoma cells.

FIG. 9: Experimental plan for in vivo assessment of AChRb1-CAAR T cells. 200,000 hybridoma cells (B3) are injected, this cell line expresses an AChR-beta1-reactive antibody. There are 2 groups (n=6 animals each): control T cells and AChR-beta1-CAAR T cells. Injection of 10 million human T cells is carried out on day 3 after injection of the hybridoma cells. Bioluminiscence imaging quantification (for the detection of in vivo killing) is carried out. Quantification of anti-Beta3 serum levels by ELISA or RIA (detection of the reduction in circulating antibodies) is carried out, as is post mortem analysis of the treated animals (off-target toxicity).

### EXAMPLES

The invention is demonstrated by way of the examples disclosed below. The examples provide technical support for a more detailed description of potentially preferred, non-limiting embodiments of the invention.

The present invention employs a CAAR with a receptor fragment of AChR instead of an antibody portion typically used in a CAR-T approach (FIG. 1). When an AChR autoantibody-producing B cell binds to the CAAR-T construct, the binding leads to an activation of the T cell, the formation of an ‘immunological synapse’ with the release of toxic mediators, which leads to the lysis of the disease-specific B cell (FIG. 1, left side). Other B cells (e.g. those with antibodies not binding the AChR) are spared from depletion (FIG. 1, right side). In FIG. 2, a schematic diagram of the alpha-1 subunit of nicotinic AChR (nAChR) is presented, in addition to a comparison with the alpha-1 subunit of nAChR with AChRa1-CAAR.

The inventors first created a construct to show the feasibility of the approach in treating myasthenia gravis (refer schematic in FIG. 2). This construct is based on the backbone of a CAR-T vector, which contains the immunologically most important part of AChR, the so-called “main immunogenic region” of the alpha-1 subunit of nAChR, instead of the antibody fragment common in CAR-T cells (Tzartos 1981). For the construct, the amino acid sequence is provided in SEQ ID NO 18.

This CAAR-T construct was lentivirally transduced by the shuttle vector FUGW (Addgene #14883, Lois et al. 2002) into primary human T cells with transduction rates of 25-60% (FIG. 3A) and expanded 400-fold over 8-12 days. As shown in FIG. 3A, the expression and functionality of the AChR-CAAR construct AchRa1-CAAR has been demonstrated in human T cells. A flow cytometric analysis of primary human T cells transduced with AChRa1-CAAR shows a surface expression of 37.6% (stained with commercial antibody mAb35 directed against AChR), which indicates a high transduction rate.

The function of the CAAR T cells was further tested in an in vitro assay. The assay determined whether contact of the CAAR T-cell with an anti-AChR antibody leads to activation of the CAAR T-cell, which is quantified by interferon-y measurement. For this purpose, an ELISA plate is coated with the antibody mAb35 (Tzartos 1981), which is widely used in myasthenia gravis research. We chose this antibody in the proof-of-concept phase because the antibody described in the 1980s is one of the best characterized antibodies and shows characteristic effects for myasthenia gravis in a variety of in vitro and in vivo models. After coating the plates with mAb35, CAAR T cells or control T cells were added. The activation of CAAR T cells leads to a release of interferon-y, which is measured in the supernatant. FIG. 4 (B) shows that only in the combination of an AChRa1 antibody (mAb35) and an AChRa1-CAAR-T cell (blue) a massive release of interferon-y occurs, but not when coated with control antibodies (mGo) or incubated with CAAR T cells that bind NMDAR antibodies or with unmodified human T cells. Activated AChRa1-CAAR T cells released, with great specificity, large amounts of interferon-y, indicating activation of the cytotoxic T cell in response to the pathogenic anti-AChR autoantibody. None of the control conditions showed interferon-y release, indicating the surprisingly good reactivity to the pathogenic antibody and high specificity.

Additional experiments were carried out to assess activation of CAAR T cells by AChRa1 antibodies on the surface of model target cells. For this test the inventors used the model of an AChRa1 receptor antibody-producing hybridoma cell (ATCC TIB.175 cells). These hybridoma cells express the pathogenic antibody mAb35 on their surface and represent a model system for an autoantibody-producing pathogenic B cell, as found in MG. After co-culture of hybridoma cells with AChRa1-CAAR T cells, the inventors observed a strong activation of CAAR T cells with corresponding cytotoxicity (FIG. 4A). Even in a low E:T ratio, strong and specific cytolysis of the anti-AChR producing model B cells took place. Control CAAR T cells with an ATD-S1-S2 (subunits of the NMDAR1 extracellular domain) autoantigenic portion in place of the AChR autoantigen showed no cytotoxic effect against TIB.175 cells, but were effective as a positive control against Nalm6 01003-102 cells (expressing an anti-NR1 antibody) (FIG. 4B).

AchRa1-CAAR T (or other CAAR-T cells) were incubated with Nalm6 #mGo53 target cells, which express the control antibody #mGo53 on their surface. AchRa1-CAAR T cells showed no relevant cytotoxic effect against Nalm6 #mGo53 cells. In particular, at low E:T ratios, where a strong specific killing of AchRa1-CAAR T cells against mAb35 hybridoma cells was observed (4a), no off-target toxicity against Nalm6 #mGo53 cells was observed.

Additional experiments were conducted with alternative CAAR constructs comprising as the autoantigenic portion the gamma subunit of the nicotinic acetylcholine receptor (nAChR), in particular the gamma subunit isoform 1 (SEQ ID NO: 5; AChRγ-CAAR), and in another experiment the combination of extracellular autoantigenic parts of alpha-1 isoform 1 and beta-1 isoform 1 subunits (SEQ ID NO: 11; AChRα1β1-CAAR). FIG. 5 demonstrates that good transduction and expression of these constructs was achieved.

Further experiments were carried out to investigate the proliferation of AChRa1-CAAR T cells. The inventors conducted a co-culture of AChRa1-CAAR T cells together with alpha- and with beta-specific hybridoma cells. Beforehand, CAAR T cells were stained with CellTrace™ Violet Cell Proliferation Kit. Here, a strong proliferation of CAAR T cells was observed when incubated with alpha-specific hybridoma cells, but not with beta-specific hybridomas. Results are presented in FIG. 6.

Following these experiments, the inventors undertook an assessment of the cytolytic activity of AChRa1- and AChRb1-CAAR T cells. AChRa1- and AChRb1-CAAR T cells deplete the respective target cells (hybridomas) within 18 h in a dose-dependent manner. Control hybridomas (8-18C5) are not targeted by CAAR T cells. Results are presented in FIG. 7.

Further experiments were conducted to investigate the expression of activation markers by the inventive CAAR T cells. After co-culture in a E:T ratio of 1:1 for 20 hours, (CD4+ and CD8+) CAAR T cells express activation markers CD25 and CD69 after co-culture with respective hybridoma cells. Results are presented in FIG. 8. Interestingly, only a few AChRb1-CAAR T cells express activation markers, but these cells still have strong cytolytic potential (see FIG. 7).

In order to assess the CAAR T cells in vivo, an experimental setup is carried out as follows. The experiment comprises injecting 200,000 hybridoma cells (B3) to NSG mice, this cell line expresses an AChR-beta1-reactive antibody. There are 2 groups (n=6 animals each), control T cells and AChR-beta1-CAAR T cells. The injection of 10 million human T cells is carried out on day 3 after injection of the hybridoma cells.

The planned readouts include bioluminiscence imaging quantification (for the detection of in vivo killing), quantification of anti-Beta3 serum levels by ELISA or RIA (for the detection of the reduction in circulating antibodies), and a post-mortem analysis of the treated animals (to assess off-target toxicity). The experimental setup is disclosed in FIG. 9.

### REFERENCES

Gilhus N E. Myasthenia Gravis. N Engl J Med. 2016 Dec. 29; 375(26):2570-2581
Gilhus N E, Tzartos S, Evoli A, Palace J, Burns T M, Verschuuren JJGM. Myasthenia gravis. Nat Rev Dis Primers. 2019 May 2; 5(1):30.
Ellebrecht C T, Bhoj V G, Nace A, et al. Reengineering chimeric antigen receptor T cells for targeted therapy of autoimmune disease. Science 2016; 353(6295)179-84
Tzartos S J, et al. Mapping of surface structures of Electrophorus acetylcholine receptor using monoclonal antibodies. J. Biol. Chem. 256: 8635-8645,1981

1. A chimeric autoantibody receptor (CAAR) polypeptide, comprising the following structure:
an extracellular domain comprising an autoantigen of a nicotinic acetylcholine receptor (nAChR) or fragment thereof,
a transmembrane domain, and
an intracellular signaling domain.

2. The chimeric autoantibody receptor (CAAR) polypeptide according to claim 1, wherein the nicotinic acetylcholine receptor (nAChR) autoantigen of the CAAR is bound by autoantibodies associated with a neuromuscular disorder.

3. The chimeric autoantibody receptor (CAAR) polypeptide according to claim 2, wherein the autoantigen of the CAAR is bound by autoantibodies in subjects with myasthenia gravis (MG), or arthrogryposis multiplex congenita (AMC) caused by diaplacental transfer of autoantibodies.

4. The chimeric autoantibody receptor (CAAR) polypeptide according to claim 1, wherein the autoantigen of the CAAR comprises an extracellular part of the nicotinic acetylcholine receptor (nAChR) or fragment thereof bound by autoantibodies.

5. The chimeric autoantibody receptor (CAAR) polypeptide according to claim 1, wherein the autoantigen of the CAAR comprises an beta-1, alpha-1, gamma, delta, or epsilon subunit, of a nicotinic acetylcholine receptor (nAChR), or an autoantigenic fragment and/or combinations thereof.

6. The chimeric autoantibody receptor (CAAR) polypeptide according to claim 1, wherein the autoantigen of the CAAR comprises or consists of a nicotinic acetylcholine receptor (nAChR) beta-1 subunit isoform 1 (SEQ ID NO: 3), beta-1 subunit isoform 2 (SEQ ID NO: 4), alpha-1 subunit isoform 1 (SEQ ID NO: 1), alpha-1 subunit isoform 2 (SEQ ID NO: 2), gamma subunit isoform 1 (SEQ ID NO: 5), gamma subunit isoform 2 (SEQ ID NO: 6), delta subunit isoform 1 (SEQ ID NO: 7), delta subunit isoform 2 (SEQ ID NO: 8), epsilon subunit (SEQ ID NO: 9), or an autoantigenic fragment and/or combination and/or variant with at least 80% sequence identity thereto.

7. The chimeric autoantibody receptor (CAAR) polypeptide according to claim 1, wherein the autoantigen of the CAAR comprises or consists of a nicotinic acetylcholine receptor (nAChR) beta-1 subunit isoform 1 according to SEQ ID NO: 3 or an autoantigenic fragment and/or variant with at least 80% sequence identity thereto.

8. The chimeric autoantibody receptor (CAAR) polypeptide according to claim 1, wherein the autoantigen of the CAAR comprises a nicotinic acetylcholine receptor (nAChR) ECD beta-1 subunit isoform 1 according to SEQ ID NO: 21 or an autoantigenic fragment and/or variant with at least 80% sequence identity thereto.

9. The chimeric autoantibody receptor (CAAR) polypeptide according to claim 1, wherein the autoantigen of the CAAR comprises or consists of a nicotinic acetylcholine receptor (nAChR) alpha-1 subunit isoform 1 according to SEQ ID NO: 1 or an autoantigenic fragment and/or variant with at least 80% sequence identity thereto.

10. The chimeric autoantibody receptor (CAAR) polypeptide according to claim 9, wherein the autoantigen of the CAAR comprises an extracellular autoantigenic part of an alpha-1 subunit isoform 1 (SEQ ID NO: 10), a combination of extracellular autoantigenic parts of alpha-1 isoform 1 and beta-1 isoform 1 subunits (SEQ ID NO: 11) or an extracellular autoantigenic part of a gamma subunit isoform 1 (SEQ ID NO: 12) of a nicotinic acetylcholine receptor (nAChR), or variant with at least 80% sequence identity thereto.

11. The chimeric autoantibody receptor (CAAR) polypeptide according to claim 1:
wherein the transmembrane domain is a CD8 alpha, CD28 or ICOS transmembrane domain;
wherein the intracellular domain comprises a CD137 (4-1BB), CD28 or ICOS co-stimulatory domain;
wherein the intracellular domain comprises a CD3 zeta chain signaling domain; and/or
wherein the nucleic acid molecule comprises additionally encodes one or more leader, linker and/or spacer polypeptides positioned N-terminally of the extracellular domain and/or between the extracellular domain and transmembrane domain and/or between the transmembrane domain and intracellular domain.

12. The chimeric autoantibody receptor (CAAR) polypeptide encoding a chimeric autoantibody receptor (CAAR) according to claim 1, wherein the nucleic acid molecule additionally comprises:
i. an extracellular domain comprising an autoantigen, comprising or consisting of a nicotinic acetylcholine receptor (nAChR) beta-1 subunit isoform 1 (SEQ ID NO: 3), beta-1 subunit isoform 2 (SEQ ID NO: 4), alpha-1 subunit isoform 1 (SEQ ID NO: 1), alpha-1 subunit isoform 2 (SEQ ID NO: 2), gamma subunit isoform 1 (SEQ ID NO: 5), gamma subunit isoform 2 (SEQ ID NO: 6), delta subunit isoform 1 (SEQ ID NO: 7), delta subunit isoform 2 (SEQ ID NO: 8), epsilon subunit (SEQ ID NO: 9), or an autoantigenic fragment and/or combination and/or variant with at least 80% sequence identity thereto
ii. optionally a linker polypeptide positioned between the extracellular domain and transmembrane domain, comprising a sequence according to SEQ ID NO 13, or a sequence with at least 80% sequence identity thereto;
iii. a CD8 alpha transmembrane domain, comprising a sequence according to SEQ ID NO 15, or a sequence with at least 80% sequence identity thereto; and
iv. an intracellular signaling domain comprising a CD137 (4-1BB) co-stimulatory domain and a CD3 zeta chain signaling domain, comprising a sequence according to SEQ ID NO 16 (CD137) and SEQ ID NO 17 (CD3z), or sequences with at least 80% sequence identity thereto.

13. The chimeric autoantibody receptor (CAAR) polypeptide encoding a chimeric autoantibody receptor (CAAR) according to claim 12, wherein the nucleic acid molecule additionally comprises:
i. an extracellular domain comprising an autoantigen, comprising or consisting of a nicotinic acetylcholine receptor (nAChR) beta-1 subunit isoform 1 according to SEQ ID NO: 3.

14. The chimeric autoantibody receptor (CAAR) polypeptide encoding a chimeric autoantibody receptor (CAAR) according to claim 13, wherein the nucleic acid molecule additionally comprises:
i. an extracellular domain comprising an autoantigen, comprising or consisting of a nicotinic acetylcholine receptor (nAChR) ECD of beta-1 subunit isoform 1 according to SEQ ID NO: 21, optionally comprising a linker.

15. The chimeric autoantibody receptor (CAAR) polypeptide encoding a chimeric autoantibody receptor (CAAR) according to claim 12, wherein the nucleic acid molecule additionally comprises:
i. an extracellular domain comprising an autoantigen, comprising or consisting of a nicotinic acetylcholine receptor (nAChR) alpha-1 subunit isoform 1 according to SEQ ID NO: 1, optionally comprising a linker.

16. A vector comprising a nucleic acid molecule encoding a chimeric autoantibody receptor (CAAR) according to claim 1.

17. A nucleic acid molecule encoding the chimeric autoantibody receptor (CAAR) polypeptide according to claim 1.

18. (canceled)

19. (canceled)

20. (canceled)

21. (canceled)

22. A genetically modified cell comprising the chimeric autoantibody receptor (CAAR) polypeptide according to claim 1.

23. The genetically modified cell according to claim 22, comprising a CAAR with an extracellular domain comprising an autoantigen, comprising a beta-1 subunit of a nicotinic acetylcholine receptor (nAChR) or an autoantigenic fragment thereof, and wherein the genetically modified cell is in combination with a second genetically modified cell, said second cell comprising a CAAR with an extracellular domain comprising an autoantigen, comprising an alpha-1, gamma, delta, or epsilon subunit of a nicotinic acetylcholine receptor (nAChR), or an autoantigenic fragment and/or combinations thereof.

24. (canceled)

25. The genetically modified cell according to claim 22,
wherein the cell comprises a CAAR with an extracellular domain comprising an autoantigen, comprising a nicotinic acetylcholine receptor (nAChR) beta-1 subunit isoform 1 according to SEQ ID NO: 3, beta-1 subunit isoform 2 according to SEQ ID NO: 4, or the ECD of beta-1 subunit isoform 1 according to SEQ ID NO: 21,
wherein said cell is in combination with a second genetically modified cell comprising a CAAR with an extracellular domain comprising an autoantigen, comprising a nicotinic acetylcholine receptor (nAChR) alpha-1 subunit isoform 1 according to SEQ ID NO: 1, alpha-1 subunit isoform 2 according to SEQ ID NO: 2, extracellular autoantigenic part of an alpha-1 subunit isoform 1 according to SEQ ID NO: 10, or a combination of extracellular autoantigenic parts of alpha-1 isoform 1 and beta-1 isoform 1 subunits according to SEQ ID NO: 11.

26. The genetically modified cell according to claim 22, wherein the cell is selected from the group consisting of a T cell, an NK cell, a macrophage and a dendritic cell.

27. A method for the treatment and/or prevention of a neuromuscular disorder associated with autoantibodies that bind a nicotinic acetylcholine receptor (nAChR), comprising administering a cell according to claim 22 to a subject in need thereof.


SEQUENCE LISTING (22 sequences)

SEQ ID NO: 1 - protein, 457 aa, Homo sapiens
MEPWPLLLLFSLCSAGLVLGSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLPLYFIVNVIIPCLLFSFLTGLVFYLPTDSGEKMTLSISVLLSLTVFLLVIVELIPSTSSAVPLIGKYMLFTMVFVIASIIITVIVINTHHRSPSTHVMPNWVRKVFIDTIPNIMFFSTMKRPSREKQDKKIFTEDIDISDISGKPGPPPMGFHSPLIKHPEVKSAIEGIKYIAETMKSDQESNNAAAEWKYVAMVMDHILLGVFMLVCIIGTLAVFAGRLIELNQQG

SEQ ID NO: 2 - protein, 482 aa, Homo sapiens
MEPWPLLLLFSLCSAGLVLGSEHETRLVAKLFKDYSSVVRPVEDHRQVVEVTVGLQLIQLINVDEVNQIVTTNVRLKQGDMVDLPRPSCVTLGVPLFSHLQNEQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLPLYFIVNVIIPCLLFSFLTGLVFYLPTDSGEKMTLSISVLLSLTVFLLVIVELIPSTSSAVPLIGKYMLFTMVFVIASIIITVIVINTHHRSPSTHVMPNWVRKVFIDTIPNIMFFSTMKRPSREKQDKKIFTEDIDISDISGKPGPPPMGFHSPLIKHPEVKSAIEGIKYIAETMKSDQESNNAAAEWKYVAMVMDHILLGVFMLVCIIGTLAVFAGRLIELNQQG

SEQ ID NO: 3 - protein, 501 aa, Homo sapiens
MTPGALLMLLGALGAPLAPGVRGSEAEGRLREKLFSGYDSSVRPAREVGDRVRVSVGLILAQLISLNEKDEEMSTKVYLDLEWTDYRLSWDPAEHDGIDSLRITAESVWLPDVVLLNNNDGNFDVALDISVVVSSDGSVRWQPPGIYRSSCSIQVTYFPFDWQNCTMVFSSYSYDSSEVSLQTGLGPDGQGHQEIHIHEGTFIENGQWEIIHKPSRLIQPPGDPRGGREGQRQEVIFYLIIRRKPLFYLVNVIAPCILITLLAIFVFYLPPDAGEKMGLSIFALLTLTVFLLLLADKVPETSLSVPIIIKYLMFTMVLVTFSVILSVVVLNLHHRSPHTHQMPLWVRQIFIHKLPLYLRLKRPKPERDLMPEPPHCSSPGSGWGRGTDEYFIRKPPSDFLFPKPNRFQPELSAPDLRRFIDGPNRAVALLPELREVVSSISYIARQLQEQEDHDALKEDWQFVAMVVDRLFLWTFIIFTSVGTLVIFLDATYHLPPPDPFP

SEQ ID NO: 4 - protein, 429 aa, Homo sapiens
MSTKVYLDLEWTDYRLSWDPAEHDGIDSLRITAESVWLPDVVLLNNNDGNFDVALDISVVVSSDGSVRWQPPGIYRSSCSIQVTYFPFDWQNCTMVFSSYSYDSSEVSLQTGLGPDGQGHQEIHIHEGTFIENGQWEIIHKPSRLIQPPGDPRGGREGQRQEVIFYLIIRRKPLFYLVNVIAPCILITLLAIFVFYLPPDAGEKMGLSIFALLTLTVFLLLLADKVPETSLSVPIIIKYLMFTMVLVTFSVILSVVVLNLHHRSPHTHQMPLWVRQIFIHKLPLYLRLKRPKPERDLMPEPPHCSSPGSGWGRGTDEYFIRKPPSDFLFPKPNRFQPELSAPDLRRFIDGPNRAVALLPELREVVSSISYIARQLQEQEDHDALKEDWQFVAMVVDRLFLWTFIIFTSVGTLVIFLDATYHLPPPDPFP

SEQ ID NO: 5 - protein, 517 aa, Homo sapiens
MHGGQGPLLLLLLLAVCLGAQGRNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALYCNVLVSPDGCIYWLPPAIFRSACSISVTYFPFDWQNCSLIFQSQTYSTNEIDLQLSQEDGQTIEWIFIDPEAFTENGEWAIQHRPAKMLLDPAAPAQEAGHQKVVFYLLIQRKPLFYVINIIAPCVLISSVAILIHFLPAKAGGQKCTVAINVLLAQTVFLFLVAKKVPETSQAVPLISKYLTFLLVVTILIVVNAVVVLNVSLRSPHTHSMARGVRKVFLRLLPQLLRMHVRPLAPAAVQDTQSRLQNGSSGWSITTGEEVALCLPRSELLFQQWQRQGLVAAALEKLEKGPELGLSQFCGSLKQAAPAIQACVEACNLIACARHQQSHFDNGNEEWFLVGRVLDRVCFLAMLSLFICGTAGIFLMAHYNRVPALPFPGDPRPYLPSPD

SEQ ID NO: 6 - protein, 465 aa, Homo sapiens
MHGGQGPLLLLLLLAVCLGAQGRNQEERLLADLMQNYDPNLRPAERDSDVVNVSLKLTLTNLISLNEREEALTTNVWIEMQWCDYRLRWDPRDYEGLWVLRVPSTMVWRPDIVLENKSQTYSTNEIDLQLSQEDGQTIEWIFIDPEAFTENGEWAIQHRPAKMLLDPAAPAQEAGHQKVVFYLLIQRKPLFYVINIIAPCVLISSVAILIHFLPAKAGGQKCTVAINVLLAQTVFLFLVAKKVPETSQAVPLISKYLTFLLVVTILIVVNAVVVLNVSLRSPHTHSMARGVRKVFLRLLPQLLRMHVRPLAPAAVQDTQSRLQNGSSGWSITTGEEVALCLPRSELLFQQWQRQGLVAAALEKLEKGPELGLSQFCGSLKQAAPAIQACVEACNLIACARHQQSHFDNGNEEWFLVGRVLDRVCFLAMLSLFICGTAGIFLMAHYNRVPALPFPGDPRPYLPSPD

SEQ ID NO: 7 - protein, 517 aa, Homo sapiens
MEGPVLTLGLLAALAVCGSWGLNEEERLIRHLFQEKGYNKELRPVAHKEESVDVALALTLSNLISLKEVEETLTTNVWIEHGWTDNRLKWNAEEFGNISVLRLPPDMVWLPEIVLENNNDGSFQISYSCNVLVYHYGFVYWLPPAIFRSSCPISVTYFPFDWQNCSLKFSSLKYTAKEITLSLKQDAKENRTYPVEWIIIDPEGFTENGEWEIVHRPARVNVDPRAPLDSPSRQDITFYLIIRRKPLFYIINILVPCVLISFMVNLVFYLPADSGEKTSVAISVLLAQSVFLLLISKRLPATSMAIPLIGKFLLFGMVLVTMVVVICVIVLNIHFRTPSTHVLSEGVKKLFLETLPELLHMSRPAEDGPSPGALVRRSSSLGYISKAEEYFLLKSRSDLMFEKQSERHGLARRLTTARRPPASSEQAQQELFNELKPAVDGANFIVNHMRDQNNYNEEKDSWNRVARTVDRLCLFVVTPVMVVGTAWIFLQGVYNQPPPQPFPGDPYSYNVQDKRFI

SEQ ID NO: 8 - protein, 502 aa, Homo sapiens
MEGPVLTLGLLAALAVCGSWGLNEEERLIRHLFQEKGYNKELRPVAHKEESVDVALALTLSNLISLGWTDNRLKWNAEEFGNISVLRLPPDMVWLPEIVLENNNDGSFQISYSCNVLVYHYGFVYWLPPAIFRSSCPISVTYFPFDWQNCSLKFSSLKYTAKEITLSLKQDAKENRTYPVEWIIIDPEGFTENGEWEIVHRPARVNVDPRAPLDSPSRQDITFYLIIRRKPLFYIINILVPCVLISFMVNLVFYLPADSGEKTSVAISVLLAQSVFLLLISKRLPATSMAIPLIGKFLLFGMVLVTMVVVICVIVLNIHFRTPSTHVLSEGVKKLFLETLPELLHMSRPAEDGPSPGALVRRSSSLGYISKAEEYFLLKSRSDLMFEKQSERHGLARRLTTARRPPASSEQAQQELFNELKPAVDGANFIVNHMRDQNNYNEEKDSWNRVARTVDRLCLFVVTPVMVVGTAWIFLQGVYNQPPPQPFPGDPYSYNVQDKRFI

SEQ ID NO: 9 - protein, 493 aa, Homo sapiens
MARAPLGVLLLLGLLGRGVGKNEELRLYHHLFNNYDPGSRPVREPEDTVTISLKVTLTNLISLNEKEETLTTSVWIGIDWQDYRLNYSKDDFGGIETLRVPSELVWLPEIVLENNIDGQFGVAYDANVLVYEGGSVTWLPPAIYRSVCAVEVTYFPFDWQNCSLIFRSQTYNAEEVEFTFAVDNDGKTINKIDIDTEAYTENGEWAIDFCPGVIRRHHGGATDGPGETDVIYSLIIRRKPLFYVINIIVPCVLISGLVLLAYFLPAQAGGQKCTVSINVLLAQTVFLFLIAQKIPETSLSVPLLGRFLIFVMVVATLIVMNCVIVLNVSQRTPTTHAMSPRLRHVLLELLPRLLGSPPPPEAPRAASPPRRASSVGLLLRAEELILKKPRSELVFEGQRHRQGTWTAAFCQSLGAAAPEVRCCVDAVNFVAESTRDQEATGEEVSDWVRMGNALDNICFWAALVLFSVGSSLIFLGAYFNRVPDLPYAPCIQP

SEQ ID NO: 10 - protein, 231 aa, Homo sapiens
MEPWPLLLLFSLCSAGLVLGSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLP

SEQ ID NO: 11 - protein, 475 aa, Homo sapiens
MEPWPLLLLFSLCSAGLVLGSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLAGSAGSAGSAGSAGSAGSAGSAGSSEAEGRLREKLFSGYDSSVRPAREVGDRVRVSVGLILAQLISLNEKDEEMSTKVYLDLEWTDYRLSWDPAEHDGIDSLRITAESVWLPDVVLLNNNDGNFDVALDISVVVSSDGSVRWQPPGIYRSSCSIQVTYFPFDWQNCTMVFSSYSYDSSEVSLQTGLGPDGQGHQEIHIHEGTFIENGQWENIHKPSRLIQPPGDPRGGREGQRQEVIFYLIIRRK

SEQ ID NO: 12 - protein, 240 aa, Homo sapiens
MHGGQGPLLLLLLLAVCLGAQGRNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALYCNVLVSPDGCIYWLPPAIFRSACSISVTYFPFDWQNCSLIFQSQTYSTNEIDLQLSQEDGQTIEWIFIDPEAFTENGEWAIQHRPAKMLLDPAAPAQEAGHQKVVFYLLIQRK

SEQ ID NO: 13 - protein, 14 aa, Artificial Sequence
  Linker
ASGGGGSGGGGSSG

SEQ ID NO: 14 - protein, 24 aa, Artificial Sequence
  Linker
AGSAGSAGSAGSAGSAGSAGSAGS

SEQ ID NO: 15 - protein, 24 aa, Artificial Sequence
  Transmembrane
IYIWAPLAGTCGVLLLSLVITLYC

SEQ ID NO: 16 - protein, 42 aa, Artificial Sequence
  Co-stimulatory
KRGRKKLLYIFKQPFMRPVQTTQEEDGCSCRFPEEEEGGCEL

SEQ ID NO: 17 - protein, 112 aa, Artificial Sequence
  Activation
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 423 aa, Artificial Sequence
  CAAR
MEPWPLLLLFSLCSAGLVLGSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLPASGGGGSGGGGSSGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 667 aa, Artificial Sequence
  CAAR
MEPWPLLLLFSLCSAGLVLGSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLAGSAGSAGSAGSAGSAGSAGSAGSSEAEGRLREKLFSGYDSSVRPAREVGDRVRVSVGLILAQLISLNEKDEEMSTKVYLDLEWTDYRLSWDPAEHDGIDSLRITAESVWLPDVVLLNNNDGNFDVALDISVVVSSDGSVRWQPPGIYRSSCSIQVTYFPFDWQNCTMVFSSYSYDSSEVSLQTGLGPDGQGHQEIHIHEGTFIENGQWENIHKPSRLIQPPGDPRGGREGQRQEVIFYLIIRRKASGGGGSGGGGSSGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 432 aa, Artificial Sequence
  CAAR
MHGGQGPLLLLLLLAVCLGAQGRNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALYCNVLVSPDGCIYWLPPAIFRSACSISVTYFPFDWQNCSLIFQSQTYSTNEIDLQLSQEDGQTIEWIFIDPEAFTENGEWAIQHRPAKMLLDPAAPAQEAGHQKVVFYLLIQRKASGGGGSGGGGSSGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 244 aa, Homo sapiens
MTPGALLMLLGALGPPLAPGVRGSEAEGRLREKLFSGYDSSVRPAREVGDRVRVSVGLILAQLISLNEKDEEMSTKVYLDLEWTDYRLSWDPAEHDGIDSLRITAESVWLPDVVLLNNNDGNFDVALDISVVVSSDGSVRWQPPGIYRSSCSIQVTYFPFDWQNCTMVFSSYSYDSSEVSLQTGLGPDGQGHQEIHIHEGTFIENGQWENIHKPSRLIQPPGDPRGGREGQRQEVIFYLIIRRK

SEQ ID NO: 22 - protein, 436 aa, Artificial Sequence
  CAAR
MTPGALLMLLGALGPPLAPGVRGSEAEGRLREKLFSGYDSSVRPAREVGDRVRVSVGLILAQLISLNEKDEEMSTKVYLDLEWTDYRLSWDPAEHDGIDSLRITAESVWLPDVVLLNNNDGNFDVALDISVVVSSDGSVRWQPPGIYRSSCSIQVTYFPFDWQNCTMVFSSYSYDSSEVSLQTGLGPDGQGHQEIHIHEGTFIENGQWENIHKPSRLIQPPGDPRGGREGQRQEVIFYLIIRRKASGGGGSGGGGSSGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR